(19) United States
(12) Patent Application Publication
(10) Pub. No.: US 2026/0174469 A1
(43) Pub. Date: Jun. 25, 2026

(54) SPINE CORRECTION APPLIANCES, AND SYSTEMS AND METHODS FOR THEIR MANUFACTURE

(71) Applicants: POLYVALOR, LIMITED PARTNERSHIP, Quebec (CA); VALORISATION HSJ, LIMITED PARTERSHIP, Quebec (CA)

(72) Inventors: Aymeric GUY, Montreal (CA); Carl-Eric AUBIN, St-Lambert (CA)

(21) Appl. No.: 19/128,745

(22) PCT Filed: Nov. 10, 2023
(86) PCT No.: PCT/CA2023/051503

§ 371 Date: May 9, 2025

Related U.S. Application Data
(60) Provisional application No. 63/424,688, filed on Nov. 11, 2022.

(51) Int. Cl. A61B 17/70; A61B 6/03; A61B 34/10

(52) U.S. Cl. CPC A61B 17/7002; A61B 6/032; A61B 2034/105

(57) ABSTRACT

Methods and system for determining a 3D digital model of spine correction appliance for a subject are provided. According to one aspect of the present methods, the 3D digital model is determined based on overcorrected positions of a plurality of vertebrae of the subject's spine. According to another aspect of the present methods, the 3D digital model is determined based on optimizing a configuration of a surface of a raw 3D digital model of the spine correction appliance considering factors indicative of at least one of a wear comfort and a safety of the spine correction appliance for the subject as well as growth-related changes of the subject's spine over a period of wearing the spine correction appliance.

### FIELD

The present technology relates broadly to the field of orthopedics; and more specifically, to spine correction appliances for subjects.

### BACKGROUND

In orthopedics, treatments of curvature disorders of a subject's spine, such as scoliosis, for example, can include surgical and non-surgical approaches. Non-surgical treatments can include applying spine corrections appliances, such as a spinal brace, that can be worn around a subject's torso.

The spinal brace, when worn by the subject, can be configured to exert pressure on the torso skin of the subject which in turn, through internal bony and soft tissues, transmits corrective forces to the misaligned vertebrae of the subject's spine, causing them to move from their initial (current) positions to target positions, which are typically associated with alignment of the subject's spinal column. This may help, for example, stop or slow down the progression of the curvature disorders until maturity of the skeletal system of the subject.

Certain methods of producing the spinal brace known in the art include (i) taking a negative cast and measurements of the subject's torso, such as manually, by an orthopedic practitioner (an orthotist, for example), or using common computer-assisted design tools and software; (ii) based on the cast and measurements, producing, for example by milling, a preform which is a positive brace mold for the spinal brace having a desired surface topography; and (iii) and thermoforming the spinal brace onto the positive brace mold. The preform and the spinal brace thus produced are shaped to cause pressure on the subject's torso, further transmitted to the subject's spine to cause the misaligned vertebrae to move towards their target positions.

Such conventional methods rely on manually designing and making the preform which are associated with certain inconveniences. For example, as it can be appreciated, the conventional methods are highly dependent on the expertise of the orthopedic practitioner which may vary between practitioners. Also, manually executed steps of the spinal brace production process may be associated with relatively long production times and increased risks of human error.

Further, spinal braces produced by conventional methods having static shapes, which do not take into account growth of the subject during the course of the orthopedic treatment. This may result in decreased biomechanical effectiveness and increasing discomfort over time, affecting the adherence of the subject to the treatment and thus reducing the overall efficacy.

Thus, there is a need in the art for methods and systems for producing spine correction appliances addressing the above-mentioned technical problems.

### SUMMARY

It is an object of the present technology to ameliorate at least some of the inconveniences present in the prior art.

The developers of the present technology have devised methods and systems for more efficient production of the spine correction appliances that would allow for more effective implementation of the orthopedic treatment.

More specifically, in accordance with at least some non-limiting embodiments of the present technology, the methods described herein include obtaining a torso 3D digital model of the subject's torso (such as a finite element model, FEM, for example) including, at least, a representation of the subject's spine, rib cage and pelvis and a current skin topography of the subject. Further, the methods include determining, based on the torso 3D digital model, overcorrected positions of the vertebrae, for example, relative to a sagittal plane of the subject, defining an overcorrected shape of the spine, which further allows determining a modulated skin topography thereof, which is representative of an inner surface of the spine correction appliance.

Thus, using such a 3D digital model can allow efficiently determining the configuration of the spine correction appliance, which can further be produced using, for example, 3D printing techniques.

Further, according to other non-limiting embodiments of the present technology, the present methods allow optimizing the configuration of the spine correction appliance, determined, for example, according to the approaches mentioned above, by considering certain parameters indicative of wear comfort and safety of the spine correction appliance, which may include, for example, contact skin pressure on the subject's torso, a clearance distance between the appliance and the pelvis of a subject, a stress value applied to a spinal anatomical component, and the like.

Also, additional non-limiting embodiments of the present methods may allow optimizing the configuration of the spine correction appliance taking into account growth-related changes of the spine of the subject during the course of the entire orthopedic treatment, further improving the wear comfort and safety of the spine correction appliance.

The spine correction appliance having the so optimized configuration can allow for improved biomechanical effectiveness over time, and improved wear comfort of the spine correction appliance by the subject, which may thus be associated with higher adherence of the subject to the treatment, thus allowing improving the overall effectiveness of the treatment.

More specifically, in accordance with a first broad aspect of the present technology, there is provided a computer-implementable method of generating a model of a spine correction appliance to be worn around a torso of a subject for correcting a misalignment in a chain of adjacent vertebrae of a spine of the subject from an initial position to a target position. The method comprises: obtaining a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtaining a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulating an initial position of a given sub-region of the plurality of sub-regions to determine a surface of the appliance 3D digital model, the modulating comprising executing an optimization algorithm, the executing comprising: iteratively optimizing the initial position of the given sub-region considering an anatomical parameter associated with the torso of the subject when wearing the spine correction appliance, thereby determining, based on the torso 3D digital model, an optimized position for the given sub-region of the surface of the raw appliance 3D digital model, determining, based on the optimized position for the given sub-region, the surface of the appliance 3D digital model; and storing data indicative of the appliance 3D digital model.

In some implementations of the method, the method further comprises determining the raw appliance 3D digital model, the determining comprising: determining, in the torso 3D digital model, for the given vertebra, a transposed position thereof, the determining comprises mirroring, in the torso 3D digital model, the respective initial position of the given vertebra relative to an anatomical sagittal plane associated with the subject; and determining, based on the transposed position of the given vertebra, within the torso 3D digital model, a modulated skin topography of the torso corresponding to the transposed position of the given vertebra, the modulated skin topography defining an inner surface of the spine correction appliance; and based on the modulated skin topography of the torso, determining the raw appliance 3D digital model of the spine correction appliance to be applied to the torso of the subject to cause the given vertebra to displace from the initial position towards the transposed position.

In some implementations of the method, the raw appliance 3D digital model is associated with a predetermined longitudinal axis; and the modulating the initial position of the given sub-region comprises modulating a respective distance of the given sub-region relative to a longitudinal axis of a cylindrical coordinate system defined around the raw appliance 3D digital model such that the longitudinal axis of the cylindrical coordinate system is aligned with the predetermined longitudinal axis of the raw appliance 3D digital model.

In some implementations of the method, the method further comprises sub-dividing the raw appliance 3D digital model in the plurality of sub-regions.

In some implementations of the method, the sub-dividing the raw appliance 3D digital model comprises: dissecting the surface of the raw appliance 3D digital model along an azimuth of the cylindrical coordinate system into a first number of sub-regions; and dissecting the surface of the raw appliance 3D digital model along the longitudinal axis of the cylindrical coordinate system into a second number of sub-regions.

In some implementations of the method, the torso 3D digital model is a finite element model (FEM).

In some implementations of the method, the anatomical parameter comprises an alignment metric that is indicative of a respective difference value between (i) a respective current position, at a given iteration of the optimization algorithm, and (ii) the respective target position of a given one of the chain of adjacent vertebrae of the spine; and the iteratively optimizing comprises minimizing the alignment metric.

In some implementations of the method, the method further comprises acquiring a treatment period for correcting the misalignment of the chain of adjacent vertebrae; determining, based on the torso 3D digital model, an updated configuration of the spine at an end of the treatment period, the updated configuration being indicative of how the given vertebral body will change in height over the treatment period; and wherein the executing the optimization algorithm comprises: iteratively optimizing the initial position of the given sub-region by minimizing the alignment metric, thereby determining, based on the torso 3D digital model including the updated configuration of the spine, the optimized position for the given sub-region of the surface of the raw appliance 3D digital model.

In some implementations of the method, the alignment metric comprises one or more anatomical parameters which are representative of an alignment of the spine.

In some implementations of the method, the minimizing the alignment metric is executed such that a safety parameter is not greater than a safety threshold, and wherein the safety parameter is one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance.

In some implementations of the method, the anatomical parameter comprises a safety parameter, the safety parameter comprising one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance; and the iteratively optimizing is executed such that the safety parameter is not greater than a safety threshold.

In some implementations of the method, the spine correction treatment comprises a plurality of stages to be implemented over the treatment period, each stage having a respective treatment interval, a given stage of the plurality of stages comprising applying, for the respective treatment interval, a respective configuration of the spine correction appliance to the torso of the subject causing at least one of the chain of adjacent vertebrae to move towards the respective target position.

In some implementations of the method, the respective target position of the given vertebra is a position thereof within the spine having a normal curvature.

In some implementations of the method, the method further comprises causing display of the appliance 3D digital model.

In some implementations of the method, the method further comprises causing the manufacturing the spine correction appliance according to the appliance 3D digital model.

In some implementations of the method, the manufacturing comprises 3D printing the spine correction appliance.

In accordance with a second broad aspect of the present technology, there is provided a system for generating a model of a spine correction appliance to be worn around a torso of a subject for correcting a misalignment in a chain of adjacent vertebrae of a spine of the subject from an initial position to a target position. The system includes at least one processor and at least one non-transitory computer-readable memory storing instruction, which, when executed by the at least one processor cause the system to: obtain a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtain a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulate an initial position of a given sub-region of the plurality of sub-regions to determine a surface of the appliance 3D digital model, by executing an optimization algorithm, the executing comprising: iteratively optimizing the initial position of the given sub-region considering an anatomical parameter associated with the torso of the subject when wearing the spine correction appliance, thereby determining, based on the torso 3D digital model, an optimized position for the given sub-region of the surface of the raw appliance 3D digital model, determine, based on the optimized position for the given sub-region, the surface of the appliance 3D digital model; and store, in the non-transitory computer-readable memory, data indicative of the appliance 3D digital model.

In some implementations of the system, the at least one processor further causes the system to determine the raw appliance 3D digital model, by: determining in the torso 3D digital model, for the given vertebra, a transposed position thereof, the determining comprises mirroring, in the torso 3D digital model, the respective initial position of the given vertebra relative to an anatomical sagittal plane associated with the subject; and determining, based on the transposed position of the given vertebra, within the torso 3D digital model, a modulated skin topography of the torso corresponding to the transposed position of the given vertebra, the modulated skin topography defining an inner surface of the spine correction appliance; and based on the modulated skin topography of the torso, determining the raw appliance 3D digital model of the spine correction appliance to be applied to the torso of the subject to cause the given vertebra to displace from the initial position towards the transposed position.

In some implementations of the system, the raw appliance 3D digital model is associated with a predetermined longitudinal axis; and to modulate the initial position of the given sub-region, the at least one processor causes the system to modulate a respective distance of the given sub-region relative to a longitudinal axis of a cylindrical coordinate system defined around the raw appliance 3D digital model such that the longitudinal axis of the cylindrical coordinate system is aligned with the predetermined longitudinal axis of the raw appliance 3D digital model.

In some implementations of the system, the at least one processor further causes the system to sub-divide the raw appliance 3D digital model in the plurality of sub-regions, wherein to sub-divide the raw appliance 3D digital model, the at least one processor causes the system to: dissect the surface of the raw appliance 3D digital model along an azimuth of the cylindrical coordinate system into a first number of sub-regions; and dissect the surface of the raw appliance 3D digital model along the longitudinal axis of the cylindrical coordinate system into a second number of sub-regions.

In some implementations of the system, the anatomical parameter comprises an alignment metric that is indicative of a respective difference value between (i) a respective current position, at a given iteration of the optimization algorithm, and (ii) the respective target position of a given one of the chain of adjacent vertebrae of the spine; and the iteratively optimizing comprises minimizing the alignment metric.

In some implementations of the system, the at least one processor further causes the system to: acquire a treatment period for correcting the misalignment of the chain of adjacent vertebrae; determine, based on the torso 3D digital model, an updated configuration of the spine at an end of the treatment period, the updated configuration being indicative of how the given vertebral body will change in height over the treatment period; and wherein the executing the optimization algorithm comprises: iteratively optimizing the initial position of the given sub-region by minimizing an alignment metric, thereby determining, based on the torso 3D digital model including the updated configuration of the spine, an optimized position for the given sub-region of the surface of the raw appliance 3D digital model.

In some implementations of the system, the alignment metric comprises one or more anatomical parameters which are representative of an alignment of the spine.

In some implementations of the system, the minimizing the alignment metric is executed such that a safety parameter is not greater than a safety threshold, and wherein the safety parameter is one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance.

In some implementations of the system, the anatomical parameter comprises a safety parameter, the safety parameter comprising one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance; and the iteratively optimizing is executed such that the safety parameter is not greater than a safety threshold.

Further, in accordance with a third broad aspect of the present technology, there is provided a computer-implemented method of generating a model of a spine correction appliance to be worn around a torso of a subject. The method is executable by a processor of a computer system. The method comprises: obtaining, by the processor, a torso 3D digital model of the torso of the subject, the torso 3D digital model being representative of (i) the spine of the subject including a plurality of vertebrae; and (ii) a current skin topography of the torso; identifying, by the processor, based on the torso 3D digital model, an initial position of a given vertebra of the plurality of vertebrae of the spine; determining, by the processor, in the torso 3D digital model, a transposed position of the given vertebra, the determining comprises mirroring, by the processor, in the torso 3D digital model, the initial position of the given vertebra relative to a reference plane associated with the subject; and determining, by the processor, based on the transposed position of the given vertebra, within the torso 3D digital model, a modulated skin topography of the torso corresponding to the transposed position of the given vertebra, the modulated skin topography defining an inner surface of the spine correction appliance; based on the modulated skin topography of the torso, determining, by the processor, an appliance 3D digital model of the spine correction appliance to be applied to the torso of the subject to cause the given vertebra to displace from the initial position towards the reference plane; and storing, by the processor, in a memory of the computer system, data indicative of the determined appliance 3D digital model.

In some implementations of the method, the obtaining the torso 3D digital model comprises generating the 3D digital model based on image data, the image data comprising: (i) a single-plane and/or biplanar radiograph representative of a skeletal system of the torso including the spine; and (ii) a skin 3D digital model including a plurality of mesh elements representative of the current skin topography of the torso; and the generating the torso 3D digital model comprises: generating, by the processor, based on the biplanar radiograph, a skeletal system 3D digital model including a plurality of mesh elements representative of a configuration of the skeletal system of the torso; and merging, by the processor, the skeletal system 3D digital model with the skin 3D digital model.

In some implementations of the method, the generating the torso 3D digital model further comprises applying a finite element analysis (FEA).

In some implementations of the method, the identifying the initial position of the given vertebra comprises identifying an initial position of a reference point of the given vertebra within the torso 3D digital model; and the determining the transposed position of the given vertebra comprises determining a mirrored position of the reference point of the given vertebra relative to the reference plane.

In some implementations of the method, wherein the spine correction appliance is configured to cause the given vertebra to displace from the initial position to a target position determined between the initial position and the transposed position.

In some implementations of the method, determining the target position comprises determining a midpoint of a line segment extending, within the coronal plane, between the reference point when the given vertebra is in the initial position and the reference point when the given vertebra is in the transposed position.

In some implementations of the method, the reference point comprises a vertebral body centroid of the given vertebra.

In some implementations of the method, the mirroring the initial position of the given vertebra further comprises adjusting the initial position of the given vertebra relative to a coronal and transverse planes associated with the subject.

In some implementations of the method, the reference, coronal, and transverse planes define a coordinate system associated with the subject, and wherein: the identifying the initial position of the given vertebra comprises identifying respective initial positions of respective center points of a left pedicle and a right pedicle of the given vertebra within the coordinate system; and the determining the transposed position of the given vertebra comprises determining respective coordinate values of the left and right pedicles by applying displacements thereto in the coordinate system, the displacements being determined in accordance with equations:

$${u}_{x L} = Wx * ( {x}_{R} - {x}_{L} ) ;$$

$${u}_{x R} = Wx * ( {x}_{L} - {x}_{R} ) ;$$

$${u}_{yL} = - Wy * ( {y}_{R} + {y}_{L} ) ;$$

$${u}_{yR} = - Wy * ( {y}_{L} + {y}_{R} ) ;$$

$${u}_{zL} = Wz * ( {z}_{R} - {z}_{L} ) ; and$$

$${u}_{zR} = Wz * ( {z}_{L} - {z}_{R} ) ;$$

where x_{R} is an X coordinate of the right pedicle in the initial position;
   x_{L} is an X coordinate of the left pedicle in the initial position;
   u_{xL} is an X displacement applied to the X coordinate of the left pedicle to obtain an X coordinate thereof in the transposed position of the given vertebra, x_{L}+u_{xL};
   u_{xR} is an X displacement applied to the X coordinate of the right pedicle to obtain an X coordinate thereof in the transposed position of the given vertebra, x_{R}+u_{xR};
   y_{R} is a Y coordinate of the right pedicle in the initial position;
   y_{L} is a Y coordinate of the left pedicle in the initial position;
   u_{yL} is a Y displacement applied to the Y coordinate of the left pedicle to obtain a Y coordinate thereof in the transposed position of the given vertebra, y_{L}+u_{yL};
   u_{yR} is a Y displacement applied to the Y coordinate of the right pedicle to obtain a Y coordinate thereof in the transposed position of the given vertebra, y_{R}+u_{yR};
   z_{R} is a Z coordinate of the right pedicle in the initial position;
   z_{L} is a Z coordinate of the left pedicle in the initial position;
   u_{zL} is a Z displacement applied to the Z coordinate of the left pedicle to obtain a Z coordinate thereof in the transposed position the given vertebra, z_{L}+u_{zL}
   u_{zR} is a Z displacement applied to the Z coordinate of the right pedicle to obtain a Z coordinate thereof in the transposed position of the given vertebra, z_{R}+u_{zR}
   Wx, Wy, and Wz are respective predetermined coefficients.

In some implementations of the method, each one of Wx, Wy, and Wz is 1.

In some implementations of the method, the method further comprises determining the target position of the given vertebra, the determining comprising determining target coordinates of the left and right pedicles of the given vertebra in the coordinate system by applying target displacements to the left and right pedicles, the target displacements being determined in accordance with equations:

$${u}_{xL}^{T} = 0.5 * {u}_{x L} ;$$

$${u}_{xR}^{T} = 0.5 * {u}_{x R} ;$$

$${u}_{yL}^{T} = - 0.5 * {u}_{yL} ;$$

$${u}_{yR}^{T} = - 0.5 * {u}_{yR} ;$$

$${u}_{zL}^{T} = 0.5 * {u}_{zL} ; and$$

$${u}_{zR}^{T} = 0. {5}^{*} {u}_{z R} .$$

In some implementations of the method, prior to the determining the transposed position of the given vertebra, the method further comprises: determining, based on the initial position of the given vertebra in the torso 3D digital model, whether the given vertebra is misaligned within the plurality of vertebrae of the spine; and wherein the determining the transposed position of the given vertebra is executed in response to determining that the given vertebra is misaligned.

In some implementations of the method, the determining the transposed position comprises determining a respective transposed position for each one of the plurality of vertebrae; and the determining the modified skin topography of the torso is based on the respective transposed positions of each one of the plurality of vertebrae.

In some implementations of the method, the reference plane is a sagittal plane associated with the subject.

In some implementations of the method, the reference plane is a symmetry plane associated with the spine.

In some implementations of the method, the reference plane is defined as being a sagittal plane associated with the subject rotated around a longitudinal axis of the sagittal plane until the sagittal plane encompasses therein a maximum curvature of the spine.

In some implementations of the method, the method further comprises, causing, by the processor, manufacture of the spine correction appliance from the determined appliance 3D digital model.

In some implementations of the method, the manufacturing comprises 3D printing the spine correction appliance.

In some implementations of the method, the method further comprises causing, by the processor, display of the determined appliance 3D digital model.

In some implementations of the method, the spine correction appliance is a spinal brace.

Further, in accordance with a fourth broad aspect of the present technology, there is provided a system for generating a model of a spine correction appliance to be worn around a torso of a subject. The system comprises a processor and a non-transitory computer-readable memory storing instructions. The processor, upon executing the instructions, is configured to: obtain a torso 3D digital model of the torso of the subject, the torso 3D digital model being representative of (i) the spine of the subject including a plurality of vertebrae; and (ii) a current skin topography of the torso; identify, based on the torso 3D digital model, an initial position of a given vertebra of the plurality of vertebrae of the spine; determine, in the torso 3D digital model, a transposed position of the given vertebra, by mirroring, in the torso 3D digital model, the initial position of the given vertebra relative to a reference plane associated with the subject; and determine, based on the transposed position of the given vertebra, within the torso 3D digital model, a modulated skin topography of the torso corresponding to the transposed position of the given vertebra, the modulated skin topography defining an inner surface of the spine correction appliance; based on the modulated skin topography of the torso, determine, an appliance 3D digital model of the spine correction appliance to be applied to the torso of the subject to cause the given vertebra to displace from the initial position towards the reference plane; and store, in the non-transitory computer-readable memory, data indicative of the determined appliance 3D digital model.

In some implementations of the system, to obtain the torso 3D digital model, the processor is configured to generate the 3D digital model based on image data, the image data comprising: (i) a biplanar radiograph representative of a skeletal system of the torso including the spine; and (ii) a skin 3D digital model including a plurality of mesh elements representative of the current skin topography of the torso; and the processor is configured to generate the torso 3D digital model by: generating, based on the biplanar radiograph, a skeletal system 3D digital model including a plurality of mesh elements representative of a configuration of the skeletal system of the torso; and merge the skeletal system 3D digital model with the skin 3D digital model.

In some implementations of the system, to generate the torso 3D digital model, the processor is further configured to apply a finite element analysis (FEA).

In some implementations of the system, to identify the initial position of the given vertebra, the processor is configured to identify an initial position of a reference point of the given vertebra within the torso 3D digital model; and to determine the transposed position of the given vertebra, the processor is configured to determine a mirrored position of the reference point of the given vertebra relative to the reference plane.

In some implementations of the system, the spine correction appliance is configured to cause the given vertebra to displace from the initial position to a target position determined between the initial position and the transposed position.

In some implementations of the system, the processor is configured to determine the target position as being a midpoint of a line segment extending, within the coronal plane, between the reference point when the given vertebra is in the initial position and the reference point when the given vertebra is in the transposed position.

In some implementations of the system, the reference point comprises a vertebral body centroid of the given vertebra.

In some implementations of the system, the mirroring the initial position of the given vertebra further comprises adjusting the initial position of the given vertebra relative to a coronal and transverse planes associated with the subject.

In some implementations of the system, the reference, coronal, and transverse planes define a coordinate system associated with the subject, and wherein: to identify the initial position of the given vertebra, the processor is further configured to identify respective initial positions of a left pedicle and a right pedicle of the given vertebra within the coordinate system; and to determine the transposed position of the given vertebra, the processor is further configured to determine respective coordinate values of the left and right pedicles by applying displacements thereto in the coordinate system, the displacements being determined in accordance with equations:

$${u}_{x L} = Wx * ( {x}_{R} - {x}_{L} ) ;$$

$${u}_{x R} = Wx * ( {x}_{L} - {x}_{R} ) ;$$

$${u}_{yL} = - Wy * ( {y}_{R} + {y}_{L} ) ;$$

$${u}_{yR} = - Wy * ( {y}_{L} + {y}_{R} ) ;$$

$${u}_{zL} = Wz * {z}_{R} - {z}_{L} ; and$$

$${u}_{zR} = Wz * ( {z}_{L} - {z}_{R} ) ;$$

where x_{R} is an X coordinate of the right pedicle in the initial position;
   x_{L} is an X coordinate of the left pedicle in the initial position;
   u_{xL} is an X displacement applied to the X coordinate of the left pedicle to obtain an X coordinate thereof in the transposed position of the given vertebra, x_{L}+u_{xL};
   u_{xR} is an X displacement applied to the X coordinate of the right pedicle to obtain an X coordinate thereof in the transposed position of the given vertebra, x_{R}+u_{xR};
   y_{R} is a Y coordinate of the right pedicle in the initial position;
   y_{L} is a Y coordinate of the left pedicle in the initial position;
   u_{yL} is a Y displacement applied to the Y coordinate of the left pedicle to obtain a Y coordinate thereof in the transposed position of the given vertebra, y_{L}+u_{yL};
   u_{yR} is a Y displacement applied to the Y coordinate of the right pedicle to obtain a Y coordinate thereof in the transposed position of the given vertebra, y_{R}+u_{yR};
   z_{R} is a Z coordinate of the right pedicle in the initial position;
   Z_{L} is a Z coordinate of the left pedicle in the initial position;
   u_{zL} is a Z displacement applied to the Z coordinate of the left pedicle to obtain a Z coordinate thereof in the transposed position the given vertebra, z_{L}+u_{zL}
   u_{zR} is a Z displacement applied to the Z coordinate of the right pedicle to obtain a Z coordinate thereof in the transposed position of the given vertebra, z_{R}+u_{zR}
   Wx, Wy, and Wz are respective predetermined coefficients.

In some implementations of the system, each one of Wx, Wy, and Wz is 1.

In some implementations of the system, the processor is further configured to determine the target position of the given vertebra by determining target coordinates of the left and right pedicles of the given vertebra in the coordinate system by applying target displacements to the left and right pedicles, the target displacements being determined in accordance with equations:

$${u}_{xL}^{T} = 0.5 * {u}_{x L} ;$$

$${u}_{xR}^{T} = 0.5 * {u}_{x R} ;$$

$${u}_{yL}^{T} = - 0.5 * {u}_{yL} ;$$

$${u}_{yR}^{T} = - 0.5 * {u}_{yR} ;$$

$${u}_{zL}^{T} = 0.5 * {u}_{zL} ; and$$

$${u}_{zR}^{T} = 0.5 * {u}_{z R} .$$

In some implementations of the system, prior to the determining the transposed position of the given vertebra, the processor is further configured to: determine, based on the initial position of the given vertebra in the torso 3D digital model, whether the given vertebra is misaligned within the plurality of vertebrae of the spine; and wherein the processor is configured to determine the transposed position of the given vertebra in response to determining that the given vertebra is misaligned.

In some implementations of the system, to determine the transposed position, the processor is configured to determine a respective transposed position for each one of the plurality of vertebrae; and the processor is further configured to determine the modified skin topography of the torso based on the respective transposed positions of each one of the plurality of vertebrae.

In some implementations of the system, the reference plane is a sagittal plane associated with the subject.

In some implementations of the system, the reference plane is a symmetry plane associated with the spine.

In some implementations of the system, the reference plane is defined as being a sagittal plane associated with the subject rotated around a longitudinal axis of the sagittal plane until the sagittal plane encompasses therein a maximum curvature of the spine.

In some implementations of the system, the processor is further configured to cause manufacture of the spine correction appliance from the determined appliance 3D digital model.

In some implementations of the system, manufacturing comprises 3D printing the spine correction appliance.

In some implementations of the system, the processor is further configured to cause display of the determined appliance 3D digital model.

In some implementations of the system, the spine correction appliance is a spinal brace.

In accordance with a fifth broad aspect of the present technology, there is provided a computer-implementable method of generating a model of a spine correction appliance to be worn around a torso of a subject for correcting a misalignment in a chain of adjacent vertebrae of a spine of the subject from an initial position to a target position. The method is executable by a processor of a computer system. The method comprises: obtaining, by the processor, a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtaining, by the processor, a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulating, by the processor, an initial position of a given sub-region of the plurality of sub-regions to determine a surface of the appliance 3D digital model, the modulating comprising executing an optimization algorithm, the executing comprising: iteratively optimizing, by the processor, the initial position of the given sub-region by minimizing an alignment metric, thereby determining, based on the torso 3D digital model, an optimized position for the given sub-region of the surface of the raw appliance 3D digital model, the alignment metric being indicative of a respective difference value between (i) a respective current position, at a given iteration of the optimization algorithm, and (ii) the respective target position of a given one of the chain of adjacent vertebrae of the spine; determining, by the processor, based on the optimized position for the given sub-region, the surface of the appliance 3D digital model; and storing, by the processor, in a memory of the computer system, data indicative of the appliance 3D digital model.

In accordance with a sixth broad aspect of the present technology, there is provided a computer-implementable method of generating a model of a spine correction appliance to be worn around a torso of a subject for correcting a misalignment in a chain of adjacent vertebrae of a spine of the subject from an initial position to a target position. The method is executable by a processor of a computer system. The method comprises: obtaining, by the processor, a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtaining, by the processor, a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulating, by the processor, an initial position of a given sub-region of the plurality of sub-regions to determine a surface of the appliance 3D digital model, the modulating comprising executing an optimization algorithm, the executing comprising: optimizing, by the processor, the initial position of the given sub-region such that a safety parameter is no greater than a safety threshold, thereby determining, based on the torso 3D digital model, an optimized position for the given sub-region of the surface of the raw appliance 3D digital model; determining, by the processor, based on the optimized position for the given sub-region, the surface of the appliance 3D digital model;

and storing, by the processor, in a memory of the computer system, data indicative of the appliance 3D digital model.

In some implementations of the method, the raw appliance 3D digital model has been determined as being representative of the current skin topography of the torso.

In some implementations of the method, the raw appliance 3D digital model has been determined based on a manually produced spine correction appliance.

In some implementations of the method, the method further comprises determining the raw appliance 3D digital model, the determining comprising: determining, by the processor, in the torso 3D digital model, for the given vertebra, a transposed position thereof, the determining comprises mirroring, by the processor, in the torso 3D digital model, the respective initial position of the given vertebra relative to an anatomical sagittal plane associated with the subject; and determining, by the processor, based on the transposed position of the given vertebra, within the torso 3D digital model, a modulated skin topography of the torso corresponding to the transposed position of the given vertebra, the modulated skin topography defining an inner surface of the spine correction appliance; and based on the modulated skin topography of the torso, determining, by the processor, the raw appliance 3D digital model of the spine correction appliance to be applied to the torso of the subject to cause the given vertebra to displace from the initial position towards the transposed position.

In some implementations of the method, the raw appliance 3D digital model is associated with a predetermined longitudinal axis; and the modulating the initial position of the given sub-region comprises modulating a respective distance of the given sub-region relative to a longitudinal axis of a cylindrical coordinate system defined around the raw appliance 3D digital model such that the longitudinal axis of the cylindrical coordinate system is aligned with the predetermined longitudinal axis of the raw appliance 3D digital model.

In some implementations of the method, the method further comprises sub-dividing the raw appliance 3D digital model in the plurality of sub-regions.

In some implementations of the method, the sub-dividing the raw appliance 3D digital model comprises: dissecting the surface of the raw appliance 3D digital model along an azimuth of the cylindrical coordinate system into a first number of sub-regions; and dissecting the surface of the raw appliance 3D digital model along the longitudinal axis of the cylindrical coordinate system into a second number of sub-regions.

In some implementations of the method, the torso 3D digital model is a finite element model (FEM).

In some implementations of the method, the method further comprises acquiring, by the processor, a treatment period for correcting the misalignment of the chain of adjacent vertebrae; determining, by the processor, based on the torso 3D digital model, an updated configuration of the spine at an end of the treatment period, the updated configuration being indicative of how the given vertebra will change in size over the treatment period; and wherein the executing the optimization algorithm comprises: iteratively optimizing, by the processor, the initial position of the given sub-region by minimizing the alignment metric, thereby determining, based on the torso 3D digital model including the updated configuration of the spine, the optimized position for the given sub-region of the surface of the raw appliance 3D digital model.

In some implementations of the method, the iteratively optimizing is executed such that a safety parameter is not greater than a safety threshold.

In some implementations of the method, the safety parameter is one or more of: a contact skin pressure on the torso of the subject; a distance between the spine correction appliance and an outer surface of the torso of the subject when the spine correction appliance is applied thereto; a minimum clearance distance between the spine correction appliance and a pelvis of the subject when the spine correction appliance is applied to the torso of the subject; a minimum clearance distance between the spine correction appliance and the breasts of the subject when the spine correction appliance is applied to the torso of the subject; smoothness parameter of the surface of the appliance 3D digital model; a maximum stress and/or strain inside the spine correction appliance.

In some implementations of the method, the alignment metric comprises an aggregate difference value between (i) respective current positions, at the given iteration of the optimization algorithm, and (ii) the respective target positions of each one of the chain of adjacent vertebrae, the aggregate difference value being determined as a Root Mean Square Error between the respective current positions and the respective target positions of each one of the chain of adjacent vertebrae.

In some implementations of the method, the alignment metric is expressed by an equation:

$$OF score = {W}_{in - brace} * [ {W}_{coronal}^{in - brace} * ( {Δ}_{CobbMT}^{in - brace} + {Δ}_{CobbTLL}^{in - brace} ) + {W}_{sagittal}^{in - brace} * ( {Δ}_{TK}^{in - brace} + {Δ}_{LL}^{in - brace} ) + {W}_{transverse}^{in - brace} * {Δ}_{Axial rot}^{in - brace} ] + {W}_{growth} * [ {W}_{coronal}^{growth} * ( {Δ}_{CobbMT}^{growth} + {Δ}_{CobbTLL}^{growth} ) + {W}_{sagittal}^{growth} * ( {Δ}_{{T K}_{}}^{growth} + {Δ}_{LL}^{growth} ) + {W}_{transverse}^{growth} * {Δ}_{Axial rot}^{growth} ] ,$$

where:
Δ^{in-brace}_{CobbMT} is a difference between values of a thoracic Cobb angle of the spine in states thereof where each one of the chain of adjacent vertebrae is in the respective current position and the respective target position thereof;
Δ^{in-brace}_{CobbTLL} is a difference between values of a lumbar Cobb angle of the spine in states thereof where each one of the chain of adjacent vertebrae is in the respective current position and the respective target position thereof;
Δ^{in-brace}_{TK} is a difference between values of a thoracic kyphosis angle of the spine in states thereof where each one of the chain of adjacent vertebrae is in the respective current position and the respective target position thereof;
Δ^{in-brace}LL is a difference between values of a lumbar lordosis angle of the spine in states thereof where each one of the chain of adjacent vertebrae is in the respective initial current and the respective target position thereof;
Δ^{in-brace}_{Axial rot} is an aggregate difference value of each one of the chain of adjacent vertebrae between the respective current position and the respective target position thereof in the transverse plane associated with the subject;
Δ^{growth}_{CobbMT} is a difference between values of the thoracic Cobb angle of the spine in states thereof where each one of the chain of adjacent vertebrae is in a respective future modelled position and the respective target position thereof,
   the respective future modelled position being a position of the given vertebra within the spine including growth-related changes thereof that have been simulated given an application of the spinal brace, produced in accordance with the appliance 3D digital model at the given iteration of the optimization algorithm, to the torso of the subject during a predetermined period;
Δ^{growth}_{CobbTLL} is a difference between values of the lumbar Cobb angle of the spine in states thereof where each one of the chain of adjacent vertebrae is in the respective future modelled position and the respective target position thereof;
Δ^{growth}_{TK} is a difference between values of the thoracic kyphosis angle of the spine in states thereof where each one of the chain of adjacent vertebrae is in the respective future modelled position and the respective target position thereof;
Δ^{growth}_{LL} is a difference between values of the lumbar lordosis angle of the spine in states thereof where each one of the chain of adjacent vertebrae is in the respective future modelled position and the respective target position thereof;
Δ^{growth}_{Axial rot} is an aggregate difference value of each one of the chain of adjacent vertebrae is in the respective future modelled position and the respective target position thereof in the transverse plane associated with the subject; and
W_{in-brace}, W^{in-brace}_{coronal}, W^{in-brace}_{sagittal}, W^{in-brace}ₜᵣₐₙₛᵥₑᵣₛₑ, W_{growth}, W^{growth}_{coronal}, W^{growth}_{sagittal}, and W^{growth}ₜᵣₐₙₛᵥₑᵣₛₑ are predetermined weight coefficients.

In some implementations of the method, each one of W_{in-brace}, W^{in-brace}_{coronal}, W^{in-brace}_{sagittal}, W^{in-brace}ₜᵣₐₙₛᵥₑᵣₛₑ, W_{growth}, W^{growth}_{coronal}, W^{growth}_{sagittal}, and W^{growth}ₜᵣₐₙₛᵥₑᵣₛₑ have been determined based on reference data associated with the spine of the subject.

In some implementations of the method, the alignment metric is expressed by an equation:

$$OF score = {W}_{in - brace} * ( {W}_{corMT} \vert \frac{Cobb {MT}_{in - brace}}{Cobb {MT}_{INI}} \vert + {W}_{CorTLL} \vert \frac{Cobb {TLL}_{in - brace}}{Cobb {TLL}_{INI}} \vert + {W}_{sagTK} \vert \frac{\vert {TK}_{in - brace} \vert - \vert {TK}_{N} \vert}{\vert {TK}_{INI} \vert - \vert {TK}_{N} \vert} \vert ++ {W}_{sagLL} \vert \frac{\vert {LL}_{in - brace} \vert - \vert {LL}_{N} \vert}{\vert {LL}_{INI} \vert - \vert {LL}_{N} \vert} \vert + {W}_{transverse MT} \vert \frac{AVR {MT}_{in - brace}}{AVR {MT}_{INI}} \vert + {W}_{transverse TLL} \vert \frac{AVR {TLL}_{in - brace}}{AVR {TLL}_{INI}} \vert ) ++ {W}_{growth} * ( {W}_{corMT} \vert \frac{Cobb {MT}_{growth}}{{CobbMT}_{INI}} \vert + {W}_{corTLL} \vert \frac{{CobbTLL}_{growth}}{{CobbTLL}_{INI}} \vert + {W}_{sagTK} \vert \frac{\vert {TK}_{growth} \vert - \vert {TK}_{N} \vert}{\vert {TK}_{INI} \vert - \vert {TK}_{N} \vert} \vert ++ {W}_{sagLL} \vert \frac{\vert {LL}_{growth} \vert - \vert {LL}_{N} \vert}{\vert {LL}_{INI} \vert - \vert {LL}_{N} \vert} \vert + {W}_{transverse MT} \vert \frac{AVR {MT}_{growth}}{AVR {MT}_{INI}} \vert + {W}_{transverse TLL} \vert \frac{AVR {TLL}_{growth}}{AVR {TLL}_{INI}} \vert ) ,$$

where Cobb MT_{in-brace} is a value of a thoracic Cobb angle of the spine in a state thereof where each vertebra of the chain of adjacent vertebrae is in the respective current position;
Cobb MT_{INI} is a value of the thoracic Cobb angle of the spine in a state thereof where each vertebra of the chain of adjacent vertebrae is in the respective initial position;
Cobb TLL_{in-brace} is a value of a lumbar Cobb angle of the spine in the state thereof where each vertebra of the chain of adjacent vertebrae of vertebrae is in the respective current position;
Cobb TLL_{INI} is a value of the lumbar Cobb angle of the spine in the state thereof where each vertebra of the chain of adjacent vertebrae is in the respective initial position;
TK_{in-brace} is a value of a thoracic kyphosis angle of the spine in the state thereof where each vertebra of the chain of adjacent vertebrae is in the respective current position;
TK_{INI} is a value of the thoracic kyphosis angle of the spine in the state thereof where each vertebra of the chain of adjacent vertebrae is in the respective initial position;
TK_{N} is a value penalizing TK_{in-brace} if TK_{in-brace} is outside a predetermined thoracic kyphosis angle normal range;
LL_{in-brace} is a value of a lumbar lordosis angle of the spine in the state thereof where each vertebra of the chain of adjacent vertebrae is in the respective current position;
LL_{INI} is a value of the lumbar lordosis angle of the spine in the state thereof where each vertebra of the chain of adjacent vertebrae is in the respective initial position;
LL_{N} is a value penalizing LL_{in-brace} if LL_{in-brace} is outside a predetermined lumbar lordosis angle normal range;
AVR MT_{in-brace} is a value of an axial rotation angle of an apical vertebra of a main thoracic segment of the spine in the state thereof where each vertebra of the chain of adjacent vertebrae is in the respective current position;
AVR MT_{INI} is a value of the axial rotation angle of the apical vertebra of the main thoracic segment of the spine in the state thereof where each vertebra of the chain of adjacent vertebrae is in the respective initial position;
AVR TLL_{in-brace} is a value of an axial rotation angle of an apical vertebra of a thoracolumbar segment of the spine in the state thereof where each vertebra of the chain of adjacent vertebrae is in the respective current position;
AVR TLL_{INI} is a value of the axial rotation angle of the apical vertebra of the thoracolumbar segment of the spine in the state thereof where each vertebra of the chain of adjacent vertebrae is in the respective initial position;
Cobb MT_{growth} is a value of the thoracic Cobb angle of the spine in the state thereof where each vertebra of the chain of adjacent vertebrae is in a respective future modelled position,
   the respective future modelled position being a position of the given vertebra within the spine including growth-related changes thereof that have been simulated given an application of the spinal brace, produced in accordance with the appliance 3D digital model at the given iteration of the optimization algorithm, to the torso of the subject during a predetermined period;
Cobb TLL_{growth} is a value of the lumbar Cobb angle of the spine in the state thereof where each vertebra of the chain of adjacent vertebrae is in the respective future modelled position;
TK_{growth} is a value of the thoracic kyphosis angle of the spine in the state thereof where each vertebra of the chain of adjacent vertebrae is in the respective future modelled position;
LL_{growth} is a value of the lumbar lordosis angle of the spine in the state thereof where each vertebra of the chain of adjacent vertebrae is in the respective future modelled position;
AVR MT_{growth} is a value of the axial rotation angle of the apical vertebra of the main thoracic segment of the spine in the state thereof where each vertebra of the chain of adjacent vertebrae is in the respective future modelled position;
AVR TLL_{growth} is a value of the axial rotation angle of the apical vertebra of a thoracolumbar segment of the spine in the state thereof where each vertebra of the chain of adjacent vertebrae is in the respective future modelled position; and
W_{in-brace}, W_{corMT}, W_{corTLL}, W_{sagTK}, W_{sagLL}, W_{transverse MT}, W_{transverse TLL}, and W_{growth}, are predetermined weight coefficients.

In some implementations of the method, each one of in-brace, W_{corMT}, W_{corTLL}, W_{sagTK}, W_{sagLL}, W_{transverse MT}, W_{transverse TLL}, and W_{growth} have been determined based on reference data associated with the spine of the subject.

In some implementations of the method, the optimization algorithm comprises a surrogate optimization algorithm.

In some implementations of the method, the spine correction treatment comprises a plurality of stages to be implemented over the treatment period, each stage having a respective treatment interval, a given stage of the plurality of stages comprising applying, for the respective treatment interval, a respective configuration of the spine correction appliance to the torso of the subject causing at least one of the chain of adjacent vertebrae to move towards the respective target position.

In some implementations of the method, the respective treatment interval has an equal duration for each one of the plurality of stages.

In some implementations of the method, the respective target position of the given vertebra is a position thereof within the spine having a normal curvature.

In some implementations of the method, the raw appliance 3D digital model comprises a plurality of mesh elements representative of a surface of the spine correction appliance.

In some implementations of the method, the method further comprises causing, by the processor, display of the appliance 3D digital model.

In some implementations of the method, the method further comprises causing, by the processor, the manufacturing the spine correction appliance according to the appliance 3D digital model.

In some implementations of the method, the manufacturing comprises 3D printing the spine correction appliance.

In some implementations of the method, the spine correction appliance is a spinal brace.

In accordance with a seventh broad aspect of the present technology, there is provided a system for generating a model of a spine correction appliance to be worn around a torso of a subject for correcting a misalignment in a chain of adjacent vertebrae of a spine of the subject from an initial position to a target position. The system includes a non-transitory computer-readable memory storing instruction, and a processor, which, upon executing the instructions, is configured to: obtain a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtain a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulate an initial position of a given sub-region of the plurality of sub-regions to determine a surface of the appliance 3D digital model, by executing an optimization algorithm, the executing comprising: iteratively optimizing, by the processor, the initial position of the given sub-region by minimizing an alignment metric, thereby determining, based on the torso 3D digital model, an optimized position for the given sub-region of the surface of the raw appliance 3D digital model, the alignment metric being indicative of a respective difference value between (i) a respective current position, at a given iteration of the optimization algorithm, and (ii) the respective target position of a given one of the chain of adjacent vertebrae of the spine; determine, based on the optimized position for the given sub-region, the surface of the appliance 3D digital model; and store, in the non-transitory computer-readable memory, data indicative of the appliance 3D digital model.

In accordance with an eighth broad aspect of the present technology, there is provided a system for generating a model of a spine correction appliance to be worn around a torso of a subject for correcting a misalignment in a chain of adjacent vertebrae of a spine of the subject from an initial position to a target position. The system includes a non-transitory computer-readable memory storing instruction, and a processor, which, upon executing the instructions, is configured to: obtain a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin topography of the torso; obtain a raw appliance 3D digital model of the spine correction appliance, the raw appliance 3D digital model, the raw appliance 3D digital model having been determined such that the spine correction appliance, manufactured according thereto and worn by the subject, causes at least one of the chain of adjacent vertebrae to displace from the respective initial position to the respective target position thereof; the raw appliance 3D digital model being sub-divided into a plurality of sub-regions; modulate an initial position of a given sub-region of the plurality of sub-regions to determine a surface of the appliance 3D digital model, by executing an optimization algorithm, the executing comprising: optimizing the initial position of the given sub-region such that a safety parameter is no greater than a safety threshold, thereby determining, based on the torso 3D digital model, an optimized position for the given sub-region of the surface of the raw appliance 3D digital model; determine, based on the optimized position for the given sub-region, the surface of the appliance 3D digital model; and store, in the non-transitory computer-readable memory, data indicative of the appliance 3D digital model.

In some implementations of the system, the raw appliance 3D digital model has been determined as being representative of the current skin topography of the torso.

In some implementations of the system, the raw appliance 3D digital model has been determined based on a manually produced spine correction appliance.

In some implementations of the system, the processor is further configured to determine the raw appliance 3D digital model, by: determining in the torso 3D digital model, for the given vertebra, a transposed position thereof, the determining comprises mirroring, in the torso 3D digital model, the respective initial position of the given vertebra relative to an anatomical sagittal plane associated with the subject; and determining, based on the transposed position of the given vertebra, within the torso 3D digital model, a modulated skin topography of the torso corresponding to the transposed position of the given vertebra, the modulated skin topography defining an inner surface of the spine correction appliance; and based on the modulated skin topography of the torso, determining the raw appliance 3D digital model of the spine correction appliance to be applied to the torso of the subject to cause the given vertebra to displace from the initial position towards the transposed position.

In some implementations of the system, the raw appliance 3D digital model is associated with a predetermined longitudinal axis; and the processor is configured to modulate the initial position of the given sub-region comprises by modulating a respective distance of the given sub-region relative to a longitudinal axis of a cylindrical coordinate system defined around the raw appliance 3D digital model such that the longitudinal axis of the cylindrical coordinate system is aligned with the predetermined longitudinal axis of the raw appliance 3D digital model.

In some implementations of the system, the processor is further configured to sub-divide the raw appliance 3D digital model in the plurality of sub-regions.

In some implementations of the system, the processor is configured to sub-divide the raw appliance 3D digital model by: dissecting the surface of the raw appliance 3D digital model along an azimuth of the cylindrical coordinate system into a first number of sub-regions; and dissecting the surface of the raw appliance 3D digital model along the longitudinal axis of the cylindrical coordinate system into a second number of sub-regions.

In some implementations of the system, the torso 3D digital model is a finite element model (FEM).

In some implementations of the system, the processor is further configured to: acquire a treatment period for correcting the misalignment of the chain of adjacent vertebrae; determine, based on the torso 3D digital model, an updated configuration of the spine at an end of the treatment period, the updated configuration being indicative of how the given vertebra will change in size over the treatment period; and wherein the executing the optimization algorithm comprises: iteratively optimizing, by the processor, the initial position of the given sub-region by minimizing an alignment metric, thereby determining, based on the torso 3D digital model including the updated configuration of the spine, an optimized position for the given sub-region of the surface of the raw appliance 3D digital model.

In some implementations of the system, the iteratively optimizing is executed such that a safety parameter is not greater than a safety threshold.

In some implementations of the system, the safety parameter is one or more of: a contact skin pressure on the torso of the subject; a distance between the spine correction appliance and an outer surface of the torso of the subject when the spine correction appliance is applied thereto; a minimum clearance distance between the spine correction appliance and a pelvis of the subject when the spine correction appliance is applied to the torso of the subject; a minimum clearance distance between the spine correction appliance and the breasts of the subject when the spine correction appliance is applied to the torso of the subject; smoothness parameter of the surface of the appliance 3D digital model; a maximum stress and/or strain inside the spine correction appliance.

In some implementations of the system, the alignment metric comprises an aggregate difference value between (i) respective current positions, at the given iteration of the optimization algorithm, and (ii) the respective target positions of each vertebra of the chain of adjacent vertebrae, the aggregate difference value being determined as a Root Mean Square Error between the respective current positions and the respective target positions of each vertebra of the chain of adjacent vertebrae.

In some implementations of the system, the alignment metric is expressed by an equation:

$$OF score = {W}_{in - brace} * [ {W}_{coronal}^{in - brace} * ( {Δ}_{CobbMT}^{in - brace} + {Δ}_{CobbTLL}^{in - brace} ) + {W}_{sagittal}^{in - brace} * ( {Δ}_{TK}^{in - brace} + {Δ}_{LL}^{in - brace} ) + {W}_{transverse}^{in - brace} * {Δ}_{Axial rot}^{in - brace} ] + {W}_{growth} * [ {W}_{coronal}^{growth} * ( {Δ}_{CobbMT}^{growth} + {Δ}_{CobbTLL}^{growth} ) + {W}_{sagittal}^{growth} * ( {Δ}_{TK}^{growth} + {Δ}_{LL}^{growth} ) + {W}_{transverse}^{growth} * {Δ}_{Axial rot}^{growth} ] ,$$

where:
Δ^{in-brace}_{CobbMT} is a difference between values of a thoracic Cobb angle of the spine in states thereof where each vertebra of the chain of adjacent vertebrae is in the respective current position and the respective target position thereof;
Δ^{in-brace}_{CobbTLL} is a difference between values of a lumbar Cobb angle of the spine in states thereof where each vertebra of the chain of adjacent vertebrae is in the respective current position and the respective target position thereof;
Δ^{in-brace}_{TK} is a difference between values of a thoracic kyphosis angle of the spine in states thereof where each vertebra of the chain of adjacent vertebrae is in the respective current position and the respective target position thereof;
Δ^{in-brace}_{LL} is a difference between values of a lumbar lordosis angle of the spine in states thereof where each vertebra of the chain of adjacent vertebrae is in the respective initial current and the respective target position thereof;
Δ^{in-brace}_{Axial rot} is an aggregate difference value of each vertebra of the chain of adjacent vertebrae between the respective current position and the respective target position thereof in the transverse plane associated with the subject;
Δ^{growth}_{CobbMT} is a difference between values of the thoracic Cobb angle of the spine in states thereof where each vertebra of the chain of adjacent vertebrae is in a respective future modelled position and the respective target position thereof,
   the respective future modelled position being a position of the given vertebra within the spine including growth-related changes thereof that have been simulated given an application of the spinal brace, produced in accordance with the appliance 3D digital model at the given iteration of the optimization algorithm, to the torso of the subject during a predetermined period;
Δ^{growth}_{CobbTLL} is a difference between values of the lumbar Cobb angle of the spine in states thereof where each vertebra of the chain of adjacent vertebrae is in the respective future modelled position and the respective target position thereof;
Δ^{growth}_{TK} is a difference between values of the thoracic kyphosis angle of the spine in states thereof where each vertebra of the chain of adjacent vertebrae is in the respective future modelled position and the respective target position thereof;
Δ^{growth}_{LL} is a difference between values of the lumbar lordosis angle of the spine in states thereof where each vertebra of the chain of adjacent vertebrae is in the respective future modelled position and the respective target position thereof;
Δ^{growth}_{Axial rot} is an aggregate difference value of each vertebra of the chain of adjacent vertebrae between the respective future modelled position and the respective target position thereof in the transverse plane associated with the subject; and
W^{in-brace}, W^{in-brace}_{coronal}, W^{in-brace}_{sagittal}, W^{in-brace}ₜᵣₐₙₛᵥₑᵣₛₑ, W_{growth}, W^{growth}_{coronal}, W^{growth}_{sagittal}, and W^{growth}ₜᵣₐₙₛᵥₑᵣₛₑ are predetermined weight coefficients.

In some implementations of the system, each one of W^{in-brace}, W^{in-brace}_{coronal}, W^{in-brace}_{sagittal}, W^{in-brace}ₜᵣₐₙₛᵥₑᵣₛₑ, W_{growth}, W^{growth}_{coronal}, W^{growth}_{sagittal}, and W^{growth}ₜᵣₐₙₛᵥₑᵣₛₑ have been determined based on reference data associated with the spine of the subject.

In some implementations of the system, the alignment metric is expressed by an equation:

$$OF score = {W}_{in - brace} * ( {W}_{c o r M T} \vert \frac{Cobb {MT}_{in - brace}}{Cobb {MT}_{INI}} \vert + {W}_{corTLL} \vert \frac{Cobb {TLL}_{in - brace}}{Cobb {TLL}_{INI}} \vert + {W}_{sagTK} \vert \frac{\vert {TK}_{in - brace} \vert - \vert {TK}_{N} \vert}{\vert {TK}_{INI} \vert - \vert {TK}_{N} \vert} \vert + {W}_{sagLL} \vert \frac{\vert {LL}_{in - brace} \vert - \vert {LL}_{N} \vert}{\vert {LL}_{INI} \vert - \vert {LL}_{N} \vert} \vert + {W}_{transverse MT} \vert \frac{AVR {MT}_{in - brace}}{AVR {MT}_{INI}} \vert + {W}_{transverseTLL} \vert \frac{AVR {TLL}_{in - brace}}{AVR {TLL}_{INI}} \vert ) + {W}_{growth} * ( {W}_{corMT} \vert \frac{Cobb {MT}_{growth}}{{CobbMT}_{INI}} \vert + {W}_{corTLL} \vert \frac{C o b b T L {L}_{growth}}{C o b b T L {L}_{INI}} \vert + {W}_{sagTK} \vert \frac{\vert {TK}_{growth} \vert - \vert {TK}_{N} \vert}{\vert {TK}_{INI} \vert - \vert {TK}_{N} \vert} \vert + {W}_{s a g L L} \vert \frac{\vert {LL}_{growth} \vert - \vert {LL}_{N} \vert}{\vert {LL}_{INI} \vert - \vert {LL}_{N} \vert} \vert + {W}_{transverse MT} \vert \frac{AVR {MT}_{growth}}{AVR {MT}_{INI}} \vert + {W}_{transverse TLL} \vert \frac{AVR {TLL}_{growth}}{AVR {TLL}_{N}} \vert ) ,$$

where Cobb MT_{in-brace} is a value of a thoracic Cobb angle of the spine in a state thereof where each vertebra of the chain of adjacent vertebrae is in the respective current position;
Cobb MT_{INI} is a value of the thoracic Cobb angle of the spine in a state thereof where each vertebra of the chain of adjacent vertebrae is in the respective initial position;
Cobb TLL_{in-brace} is a value of a lumbar Cobb angle of the spine in the state thereof where each vertebra of the chain of adjacent vertebrae of vertebrae is in the respective current position;
Cobb TLL_{INI} is a value of the lumbar Cobb angle of the spine in the state thereof where each vertebra of the chain of adjacent vertebrae is in the respective initial position;
TK_{in-brace} is a value of a thoracic kyphosis angle of the spine in the state thereof where each vertebra of the chain of adjacent vertebrae is in the respective current position;
TK_{INI} is a value of the thoracic kyphosis angle of the spine in the state thereof where each vertebra of the chain of adjacent vertebrae is in the respective initial position;
TK_{N} is a value penalizing TK_{in-brace} if TK_{in-brace} is outside a predetermined thoracic kyphosis angle normal range;
LL_{in-brace} is a value of a lumbar lordosis angle of the spine in the state thereof where each vertebra of the chain of adjacent vertebrae is in the respective current position;
LL_{INI} is a value of the lumbar lordosis angle of the spine in the state thereof where each vertebra of the chain of adjacent vertebrae is in the respective initial position;
LL_{N} is a value penalizing LL_{in-brace} if LL_{in-brace} is outside a predetermined lumbar lordosis angle normal range;
AVR MT_{in-brace} is a value of an axial rotation angle of an apical vertebra of a main thoracic segment of the spine in the state thereof where each vertebra of the chain of adjacent vertebrae is in the respective current position;
AVR MT_{INI} is a value of the axial rotation angle of the apical vertebra of the main thoracic segment of the spine in the state thereof where each vertebra of the chain of adjacent vertebrae is in the respective initial position;
AVR TLL_{in-brace} is a value of an axial rotation angle of an apical vertebra of a thoracolumbar segment of the spine in the state thereof where each vertebra of the chain of adjacent vertebrae is in the respective current position;
AVR TLL_{INI} is a value of the axial rotation angle of the apical vertebra of the thoracolumbar segment of the spine in the state thereof where each vertebra of the chain of adjacent vertebrae is in the respective initial position;
Cobb MT_{growth} is a value of the thoracic Cobb angle of the spine in the state thereof where each vertebra of the chain of adjacent vertebrae is in a respective future modelled position,
   the respective future modelled position being a position of the given vertebra within the spine including growth-related changes thereof that have been simulated given an application of the spinal brace, produced in accordance with the appliance 3D digital model at the given iteration of the optimization algorithm, to the torso of the subject during a predetermined period;
Cobb TLL_{growth} is a value of the lumbar Cobb angle of the spine in the state thereof where each vertebra of the chain of adjacent vertebrae is in the respective future modelled position;
TK_{growth} is a value of the thoracic kyphosis angle of the spine in the state thereof where each vertebra of the chain of adjacent vertebrae is in the respective future modelled position;
LL_{growth} is a value of the lumbar lordosis angle of the spine in the state thereof where each vertebra of the chain of adjacent vertebrae is in the respective future modelled position;
AVR MT_{growth} is a value of the axial rotation angle of the apical vertebra of the main thoracic segment of the spine in the state thereof where each vertebra of the chain of adjacent vertebrae is in the respective future modelled position;
AVR TLL_{growth} is a value of the axial rotation angle of the apical vertebra of a thoracolumbar segment of the spine in the state thereof where each vertebra of the chain of adjacent vertebrae is in the respective future modelled position; and
W_{in-brace}, W_{corMT}, W_{corTLL}, W_{sagTK}, W_{sagLL}, W_{transverse MT}, W_{transverse TLL}, and W_{growth}, are predetermined weight coefficients.

In some implementations of the system, each one of _{in-brace}, W_{corMT}, W_{corTLL}, W_{sagTK}, W_{sagLL}, W_{transverse MT}, W_{transverse TLL}, and W_{growth} have been determined based on reference data associated with the spine of the subject.

In some implementations of the system, the optimization algorithm comprises a surrogate optimization algorithm.

In some implementations of the system, the spine correction treatment comprises a plurality of stages to be implemented over the treatment period, each stage having a respective treatment interval, a given stage of the plurality of stages comprising applying, for the respective treatment interval, a respective configuration of the spine correction appliance to the torso of the subject causing at least one of the chain of adjacent vertebrae to move towards the respective target position.

In some implementations of the system, the respective treatment interval has an equal duration for each one of the plurality of stages.

In some implementations of the system, the respective target position of the given vertebra is a position thereof within the spine having a normal curvature.

In some implementations of the system, the raw appliance 3D digital model comprises a plurality of mesh elements representative of a surface of the spine correction appliance.

In some implementations of the system, the processor is further configured to cause display of the appliance 3D digital model.

In some implementations of the system, the processor is configured to cause the manufacturing the spine correction appliance according to the appliance 3D digital model.

In some implementations of the system, the manufacturing comprises 3D printing the spine correction appliance.

In some implementations of the system, the spine correction appliance is a spinal brace.

Thus, in at least some non-limiting embodiments of the present technology, using a FEM of the torso of the subject may allow modelling deformable soft tissue between the skin and the skeleton of the subject, which may further allow reproducing a more realistic, biometrically accurate, inner surface of the spine correction appliance and hence increasing the effectiveness of the spine correction treatment.

In at least some non-limiting embodiments of the present technology, sub-dividing the surface of the raw appliance 3D digital model prior to the optimization process may allow for versatility of modifying surfaces of various types of spine correction appliances with a relatively high precision rate due to controlling a plurality of parameters via a specifically defined objective function. Also, such an approach may allow predetermining a desired precision rate of modifying the surface of the spine correction appliance by selecting a desired level of granularity for sub-dividing the surface of the raw appliance 3D digital model.

Also, certain non-limiting embodiments of the present technology are directed to optimizing the shape of the spine correction appliance such that the spine correction appliance thus produced would cause the misaligned vertebrae to move to their positions associated with a normal curvature of the spine which may provide for efficacy of the planned spine correction treatment.

In the context of the present specification, unless expressly provided otherwise, a computer system may refer, but is not limited to, an “electronic device”, an “operation system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented, or otherwise rendered available for use. A database may reside on the same hardware as the process that stores or makes use of the information stored in the database or it may reside on separate hardware, such as a dedicated server or plurality of servers.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Embodiments of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIGS. 1A and 1B depict schematic diagrams of a subject's torso illustrating an example of a curvature disorder of a subject's spine and a normal curvature thereof, respectively, in accordance with certain non-limiting embodiments of the present technology;

FIG. 2 depicts a schematic diagram of a spine correction appliance used for correcting the curvature disorder of the subject's spine depicted in FIG. 1A, in accordance with certain non-limiting embodiments of the present technology;

FIG. 3 depicts a schematic diagram of a mold used for thermoforming the spine correction appliance of FIG. 2, in accordance with certain non-limiting embodiments of the present technology;

FIG. 4 depicts a schematic diagram of a system for manufacturing the spine correction appliance of FIG. 2, in accordance with certain embodiments of the present technology;

FIG. 5 depicts a schematic diagram of a computing environment of the system of FIG. 4, in accordance with certain embodiments of the present technology;

FIG. 6 depicts a flowchart diagram of a first method of determining a 3D digital model of the spinal correction appliance of FIG. 2, in accordance with the non-limiting embodiments of the present technology;

FIG. 7 depicts 3D digital models of a skeletal system and a skin topography of the subject merged in a torso 3D digital model of the subject's torso depicted in FIG. 1A, in accordance with certain non-limiting embodiments of the present technology;

FIG. 8 depicts a schematic diagram of anatomical features of a given vertebra of the subject's spine, in accordance with certain non-limiting embodiments of the present technology;

FIG. 9 depicts a schematic diagram of determining, by a processor of FIG. 5, transposed positions of vertebrae of the subject's spine, in accordance with certain non-limiting embodiments of the present technology;

FIG. 10 depicts a schematic diagram of the torso 3D digital model of FIG. 7 where the 3D digital model of the skin topography has been modified, by the processor of FIG. 5, responsive to causing the vertebrae of the subject's spine to move to the transposed positions thereof, in accordance with certain non-limiting embodiments of the present technology;

FIGS. 11A and 11B depict schematic diagrams of the 3D digital model of the spinal correction appliance determined, by the processor of FIG. 5, based on the torso 3D digital model of FIG. 10, in accordance with certain non-limiting embodiments of the present technology;

FIG. 12 depicts a flowchart diagram of a second method of determining a 3D digital model of the spine correction appliance of FIG. 2, in accordance with the non-limiting embodiments of the present technology;

FIG. 13 depicts a schematic diagram for a step of sub-dividing, by the processor of FIG. 5, a surface of the 3D digital model of FIG. 11B into a plurality of sub-regions, in accordance with certain non-limiting embodiments of the present technology;

FIG. 14 depicts a schematic diagram of a step of modulating, by the processor of FIG. 5, a position of a given sub-region of the plurality of sub-regions of the 3D digital model of FIG. 13 to determine an optimized position of the given sub-region, in accordance with certain non-limiting embodiments of the present technology;

FIG. 15 depicts a schematic diagram of an optimized 3D digital model determined by optimizing, by the processor of FIG. 5, a position of each one of the plurality of sub-regions, in accordance with certain non-limiting embodiments of the present technology; and

FIGS. 16A and 16B depict coronal and sagittal views of the subject's spine for a step of determining, by the processor of FIG. 5, certain anatomical references of the subject's spine, in accordance with certain non-limiting embodiments of the present technology.

### DETAILED DESCRIPTION

Certain aspects and embodiments of the present technology are directed to methods of and systems for developing more effective and/or more efficient spine correction treatments for a subject (also referred to herein as a “patient”), which may take into account certain safety, growth-related changes of the subject's spine and/or wear comfort considerations.

Further, it should be expressly understood that, in the context of the present specification, the term “spine correction treatment” is broadly referred to any type of medical intervention aimed at correcting curvature disorders of a spine of the subject, such as using a spine correction appliance, including, without limitation, a spinal brace, a lumbar support belt, thoracis orthoses, and the like.

More specifically, certain aspects and embodiments of the present technology comprise computer-implemented methods for manufacturing the spine correction appliance for implementing the non-surgical spine correction treatment. In some aspects and embodiments of the present technology, the methods are directed to determining a configuration of the spine correction appliance based on a 3D digital model (such as a finite element model, FEM) of the subject's torso, by (i) identifying therein misaligned vertebrae of the subject's spine; (ii) determining, for each one of the misaligned vertebrae, an overcorrected position thereof in the 3D digital model, thereby determining a modulated skin topography of the subject's torso; and (iii) and based on the 3D digital model with the so determined modulated skin topography, determining a configuration of the spine correction appliance for further manufacturing thereof.

In other aspects and embodiments of the present technology, the described methods include optimizing the configuration of the spine correction appliance by considering certain parameters indicative of at least one of subject's comfort of wearing of the spine correction appliance, safety of the spine correction appliance, and growth-related changes of the subject's spine in the course of the spine correction treatment.

Certain non-limiting embodiments of the present technology may allow improving efficiency of manufacturing of the spine correction appliance. Certain non-limiting embodiments of the present technology may allow increasing effectiveness of the non-surgical treatment thus applied compared to the prior art approaches.

More specifically, the improved efficiency may be attained by determining the configuration of the spine correction appliance based on the 3D digital model allowing accurately determining the modulated skin topography of the subject's torso in real time in response to displacing, in the 3D digital model, the misaligned vertebrae of the subject's spine to the overcorrected positions thereof. Further, higher efficiency of the manufacturing process can be attained by using lesser computational resources of a processor, which, in turn, can be attained by determining, in the 3D digital model of the subject's torso, the overcorrected positions for the misaligned vertebrae as being positions thereof “mirrored” relative to an anatomical plane associated with the subject (such as at least one of sagittal, coronal, and transverse). Also, advantageously, according to certain non-limiting embodiments of the present technology, the spine correction appliance can be produced directly from the so determined 3D digital model, representative of the modulated skin topography, using, for example, 3D printing techniques, which can also increase the efficiency of the manufacturing process. More specifically, such an approach allows omitting the steps of producing a mold for the spine correction appliance and further, using the mold, thermoforming the spine correction appliance which can have time and cost advantages.

Further, the increased effectiveness of the spine correction appliance may be attained via increasing safety and/or wear comfort thereof. To that end, the present methods are directed to optimizing the configuration of the spine correction appliance, determined, for example, via the “mirroring” approach, factoring in parameters indicative of at least one of the safety and wear comfort of the spine correction appliance as well as growth-related changes of the subject's spine during the course of the treatment. By doing so, the present methods may allow increasing subject's adherence to the spine correction treatment, which may eventually translate in the increased effectiveness thereof.

### Spine Correction Treatment

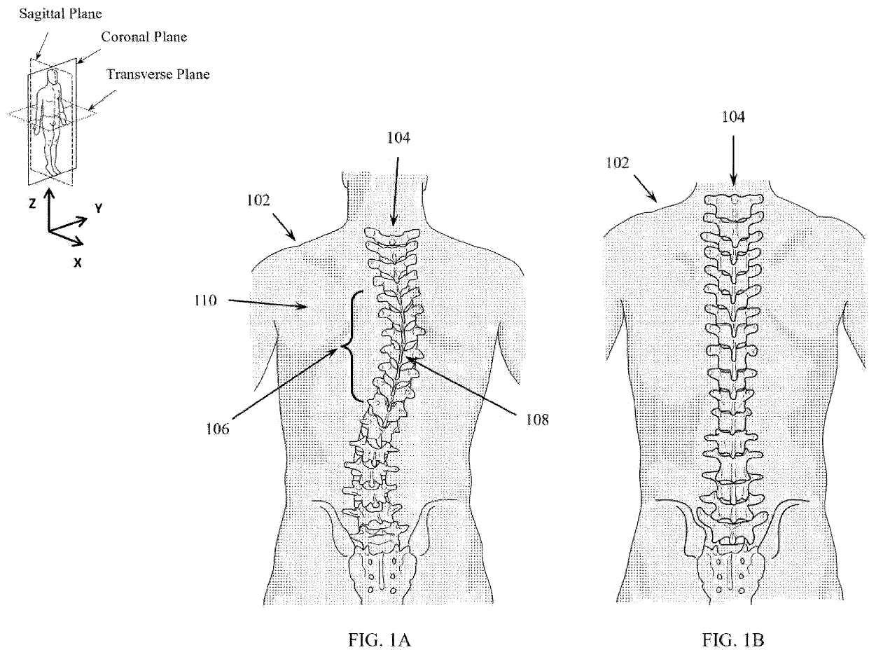

With initial reference to FIG. 1A, there is depicted a schematic diagram of a torso 102 of the subject, in accordance with certain non-limiting embodiments of the present technology.

As best shown in FIG. 1A, the torso 102 includes, inter alia, a skeletal system thereof, which includes a spine 104 including a plurality of vertebrae, covered by a torso skin 110. It should be expressly understood that the torso 102 as depicted in FIG. 1A is provided only for illustrative and explanatory purposes; therefore, certain structures, such as a ribcage, scapulae, and muscles, of the torso 102 have been omitted in FIG. 1A.

Further, as it can be appreciated from FIG. 1A, the spine 104 has a curvature disorder (also known as a spinal deformity), that is, the spine 104 includes a misaligned chain 106 of adjacent vertebrae, where at least one vertebra, such as a given vertebra 108, has been displaced, due to a birth defect, for example, within a coronal plane of the subject, from a position associated with a normal curvature of the spine 104, which is depicted, as an example, in FIG. 1B, in accordance with certain non-limiting embodiments of the present technology.

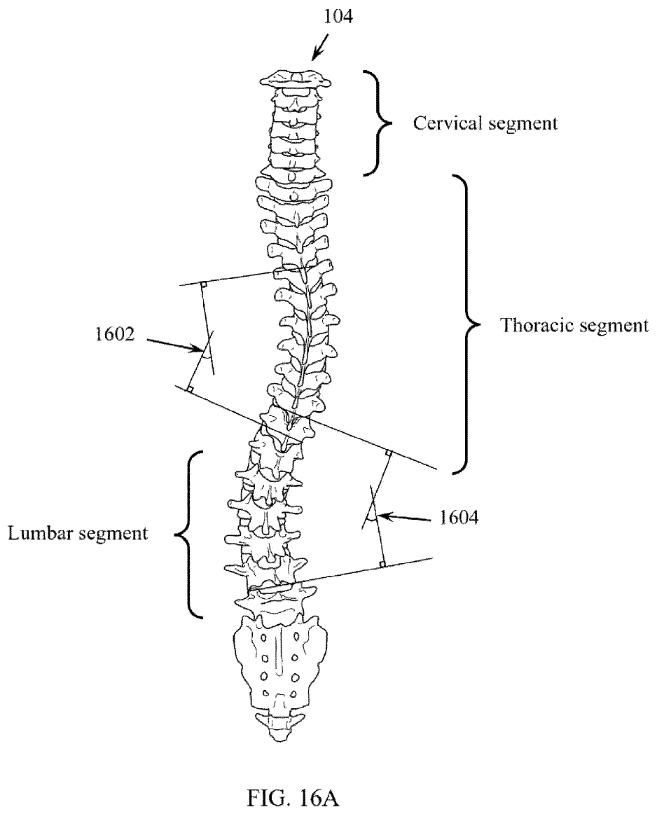

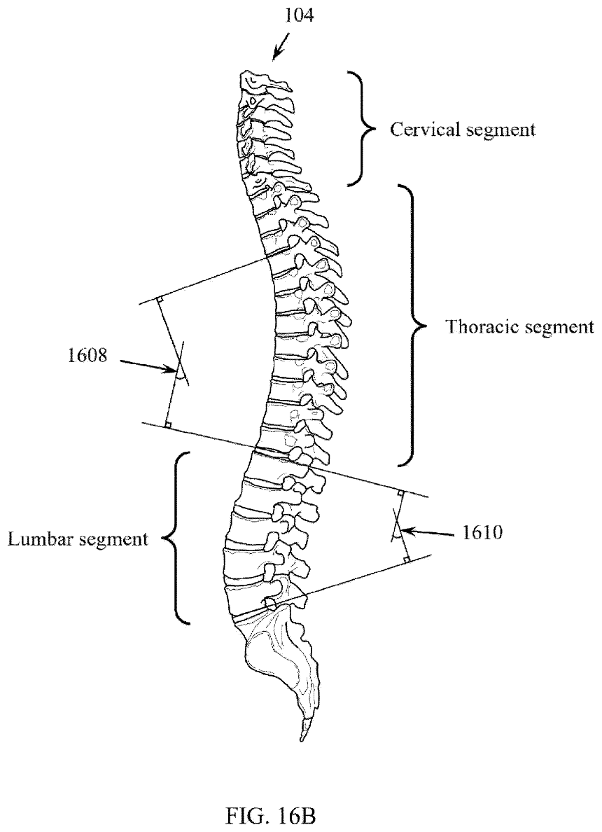

In the context of the present specification the term “normal curvature” of the spine 104 denotes a configuration of the spine in which the vertebrae are considered to be aligned, such as when thoracic and lumbar Cobb angles (such as a thoracic Cobb angle 1602 and a lumbar Cobb angle 1604 as depicted in FIG. 16A), a thoracic kyphosis angle, and a lumbar lordosis angle (such as a thoracic kyphosis angle 1606 and a lumbar lordosis angle 1608 as depicted in FIG. 16B) are within respective ranges predetermined for subjects of a certain age group. For example, for adults, the thoracic Cobb angle 1602 and the lumbar Cobb angle 1604 can be predetermined as being no greater than 10 degrees; and the thoracic kyphosis angle 1606 and the lumbar lordosis angle 1608 can be predetermined as being between about 20 and about 45 degrees and between about 30 and about 60 degrees, respectively.

In other non-limiting embodiments of the present technology, the normal curvature of the spine 104 can correspond to a configuration having (i) the thoracic Cobb angle 1602 and (ii) the lumbar Cobb angle 1604 as mentioned in the example above, and (iii) the thoracic kyphosis angle 1606 within a predetermined thoracic kyphosis angle normal range, such as from about 20 to about 40 degrees; and (iv) the lumbar lordosis angle 1608 being within a predetermined lumbar lordosis angle range, such as from about 30 to about 60 degrees.

A type of the curvature disorder of the spine 104 of the subject to which embodiments of the present technology can be applied is not limited. In one example, as depicted in FIG. 1A, when the given vertebra 108 is displaced from the normal curvature, also referred to herein as an “aligned position”, in the coronal plane associated with the subject; the curvature disorder may comprise scoliosis. In another example (not depicted), when the given vertebra 108 is displaced from the aligned position within a sagittal plane, depending on a section of the spine 104 where the given vertebra 108 is disposed, the curvature disorder may comprise at least one of a kyphosis and lordosis. Other examples (also not depicted) of the curvature disorder of the spine envisioned by the present technology, can include a flatback and swayback syndromes. Yet other example (also not depicted) of the curvature disorder of the spine 104 may include deviation of at least one vertebra, such as the given vertebra 108, from the position thereof associated with the normal curvature of the spine 104 in the transverse plane associated with the subject. In other words, the given vertebra 108 can be axially rotated in the transverse plane causing the curvature disorder of the spine 104. It should be noted that the above list of types for the curvature disorder of the spine 104 is not exhaustive and can include other curvature disorders without departing from the scope of the present technology.

Embodiments of the present technology may also apply to various combinations of the curvature disorder of the spine 104. For example, the curvature disorder of the spine 104 of the subject may include both the scoliosis and lordosis; scoliosis and the flatback syndrome; lordosis and kyphosis; one or more of the scoliosis, lordosis, and kyphosis and the deviation of the at least one vertebra in the traverse plane associated with the subject, and the like.

Thus, for correcting such a curvature disorder of the spine 104, in accordance with various non-limiting embodiments of the present technology, a spine correction treatment can be applied to the subject. In some non-limiting embodiments of the present technology, the spine correction treatment can include applying, to the torso 102 of the subject, a spine correction appliance. It is not limited which type of the spine correction appliance can be used for the curvature disorder of the spine 104, and, depending on a type, can include, without limitation, various cervical, thoracic, lumbar, and sacral orthoses.

Thus, according to certain non-limiting embodiments of the present technology, when worn by the subject onto the torso 102, the spine correction appliance can be configured to exert a respective force onto each vertebra of the misaligned chain 106, such as the given vertebra 108, thereby causing each vertebra to move to a target position, such as a position associated with the normal curvature of the spine 104. More specifically, continuing with the example of FIG. 1A, the spine correction appliance can be configured to cause the given vertebra 108 to move, in the coronal plane, towards the sagittal plane associated with the subject (that is, rightwards in the orientation of FIG. 1A) until it reaches the aligned position thereof within the spine 104.

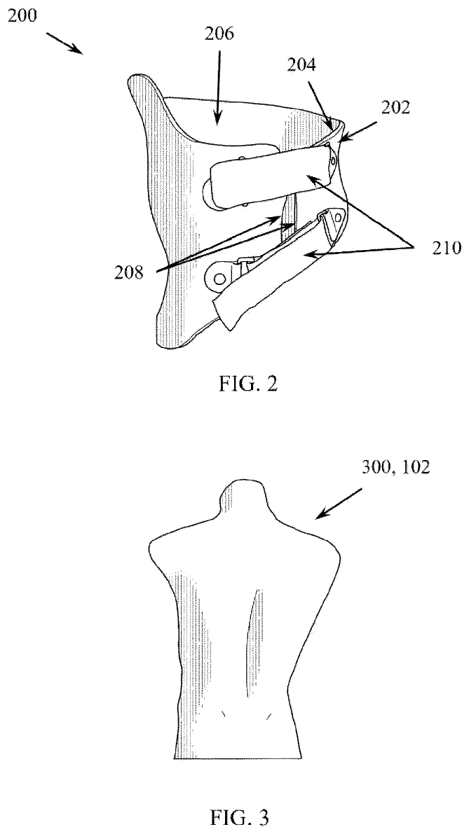

In specific non-limiting embodiments of the present technology, the spine correction appliance can include a spinal brace. With reference to FIG. 2, there is depicted a schematic diagram of a spinal brace 200 configured to correct the curvature disorder of the spine 104, such as that depicted in FIG. 1A, in accordance with certain non-limiting embodiments of the present technology.

As best seen in FIG. 2, according to certain non-limiting embodiments of the present technology, the spinal brace 200 comprises a body 201 comprising an outer surface 202 and an inner surface 204. The spinal brace 200 is configured to be wrapped around the torso 102 of the subject. When in position around the torso 102, the body 201 defines a channel 206 which is configured to receive at least a portion of the torso 102, such as the portion of the torso 102 including the misaligned chain 106 of the spine 104. However, in other non-limiting embodiments of the present technology, the channel 206 can be configured to receive a more extended portion of the torso 102, for example extending along all of the plurality of vertebrae of the spine 104 and encompassing aligned as well as misaligned portions.

Further, according to certain non-limiting embodiments of the present technology, a configuration of the spinal brace 200, including, for example, a material and a thickness, generally depends on a particular curvature disorder of the spine 104. However, as an example, in some non-limiting embodiments of the present technology, the thickness of the spinal brace 200 may be about 4.5 mm. In other non-limiting embodiments of the present technology, the thickness may be 2.5 mm, 3.0 mm, 3.5 mm, 4.0 mm, and the like. However, in yet other non-limiting embodiments of the present technology, the thickness of the spinal brace 200 can be selected as one of 5.0 mm, 10.0 mm, 20 mm, and the like. In yet other non-limiting embodiments of the present technology, the spinal brace 200 may have regions of variable thickness.

According to certain non-limiting embodiments of the present technology, the spinal brace 200 may be made of a material having certain stiffness properties, such as a polymer, including a polyethylene, for example. In other non-limiting embodiments of the present technology, the spinal brace 200 may be made of High-Density Polyethylene (HDPE). In yet other non-limiting embodiments of the present technology, the spinal brace 200 may be made of Polypropylene (PP). In yet other non-limiting embodiments of the present technology, the spinal brace 200 may be made of Polyamide Nylon 12 (PA12). Other suitable materials can also be used to form the spinal brace 200 without departing from the scope of the present technology.

Also, in some non-limiting embodiments of the present technology, depending on a desired pressure to be applied to the misaligned regions of the spine 104, the spinal brace 200 can be made of a combination of different materials. For example, the spinal brace 200 can largely be made of polyethylene with metal insets. Also, in some non-limiting embodiments of the present technology, both the outer and inner surfaces 202 and 204 can be finished with a fabric, such as cotton or nylon, as an example.

According to certain non-limiting embodiments of the present technology, the body 201 of the spinal brace 200 can have certain flexibility allowing shaping the inner surface 204 around the torso 102 of the subject. In use, the spinal brace 200 is sized to be wrapped around the torso 102 such that it extends at least partially around the torso 102. In certain embodiments, the body 201 of the spinal brace 200 extends only partially around the torso 102. In other embodiments (not shown), the body 201 is configured to extend fully around the torso 102, and may even overlap itself.

Also, it should be noted that the body 201 of the spinal brace 200 may also define and/or include certain additional elements (not depicted). For example, such additional elements can include openings or lattices configured for at least one of (i) decreasing an overall weight of the spinal brace 200, and (ii) permitting air flow to the torso 102 when the spinal brace 200 is applied. Also, the additional elements can include motifs applied along the surface of the body 201 intended for decorative or informational purposes of the spinal brace 200.

Going back to the illustrated embodiment in which the body 201 of the spinal brace 200 extends only partially around the torso 102, the body 201 has edges 208 which do not meet but remain spaced from one another to define a gap (not separately numbered) therebetween when the spinal brace 200 is worn by the subject. The edges 208 may comprise longitudinal edges of the spinal brace 200. One or more fasteners 210 are provided for removably attaching together the edges 208. In some embodiments, the fastener 210 is a strap extending across the gap and connected at either end to respective portions of the body 201 on either side of the edges 208. A distance between the edges 208 can be modulated by the fastener 210. For example, the edges 208 can be brought closer together to secure the spinal brace 200 in a desired position and with a desired tightness around the torso 102. Although the fastener 210 has been described as being a strap in certain embodiments, the type of fastener 210 is not limited, and can include, for example, without limitation, a hook-and-loop fastener, hooks, buttons, zips, laces, and the like. In specific non-limiting embodiments of the present technology, the fastener 210 can include magnet fasteners, which the developers of the present technology have considered advantageous as being simpler in use, more durable, and causing less discomfort, such as by trapping therein hair or skin of the subject, for example.

As it may become apparent, the spinal brace 200 may be designed in such a way that a topography of the inner surface 204 is configured to exert a required force to move one or more vertebrae of the misaligned chain 106 to a desired position. The desired position may be associated with the normal curvature of the spine 104 (depicted, for example, in FIG. 1B), and can be referred to as an “aligned position”. One or more such spinal braces 200 may be applied to the subject's torso in the course of a series of spinal treatments, such that each spinal brace 200 of the series will cause an incremental progression of the given vertebra to its desired position.

Thus, referring back to FIG. 1A, in order to cause the given vertebra 108 to reach the aligned position, one or more spinal braces 200 may be used. More specifically, in some non-limiting embodiments of the present technology, the spine correction treatment can include a plurality of stages, each one of which includes application of a different configuration of the spinal brace 200, each being configured to cause movement of the given vertebra 108 towards the aligned position with a certain step. However, in other non-limiting embodiments of the present technology, the spine correction treatment comprises a single stage comprising a single configuration of the spinal brace 200 configured to cause the given vertebra 108 to move to the aligned position in a single step.

Further, it is not limited how the spinal brace 200 can be manufactured; and, according to certain non-limiting embodiments of the present technology, the spinal brace 200 can be manufactured using a thermoforming process. With reference to FIG. 3, there is depicted a brace mold 300 of the torso 102 used for thermoforming the spinal brace 200, in accordance with certain non-limiting embodiments of the present technology.

More specifically, in some non-limiting embodiments of the present technology, a process of obtaining the brace mold 300 can include: (i) obtaining, such as by using a 3D scanner, a 3D scan indicative of a current skin topography of the torso skin 110 of the torso 102 of the subject; (ii) based on the 3D scan, generating a 3D digital model of the torso 102; (iii) modifying, in the 3D digital model, the current skin topography of the torso skin 110 such that the spinal brace thus produced causes the given vertebra to move towards to its aligned position, thereby defining the desired skin topography of the torso 110; and (iii) based on the 3D digital model representing the desired skin topography of the torso skin 110, causing manufacturing, such as by using computer numerical control methods, the brace mold 300.

Further, by thermoforming a precursor spinal brace (not depicted) onto the brace mold 300 and cutting off excess portions thereof, the spinal brace 200 can be manufactured.

As mentioned above, the spinal brace 200, manufactured by executing the above steps according to the prior art approaches including determination of the desired skin topography without considering the movement of the vertebrae, may have a limited efficacy due to being biomechanically suboptimal, uncomfortable (or even unsafe) affecting the subject's adherence to the spine correction treatment.

Thus, the developers of the present technology have appreciated that the desired skin topography of the torso skin 110 can be determined more accurately. In other words, unlike the prior art approaches, the present methods and systems are directed to determining the desired skin topography of the torso skin 110 within a more detailed 3D digital model of the torso 102 that would not only represent the surface of the torso 102 but would also include a 3D digital model of the skeletal system of the subject, allowing accurately reproducing the skin topography of the torso skin 110 in response to displacing therein vertebrae of the spine 104, such as the given vertebra 108 of the misaligned chain 106. For example, such a 3D digital model of the torso 102, in at least some non-limiting embodiments of the present technology, can comprise a finite element model (FEM). In other words, changes to the skin topography with movement of the vertebrae would be modeled. This may thus allow improving the safety and the wear comfort of the spinal brace 200 during the spine correction treatment. Accordingly, the subject's adherence to the so determined configuration of the spinal brace 200 can be improved, which can thus help improve the overall effectiveness of the spine correction treatment.

Advantageously according to certain non-limiting embodiments of the present technology, the spinal brace 200 can be manufactured directly from a 3D digital model thereof, by 3D printing, which may allow for a greater efficiency of the manufacturing process.

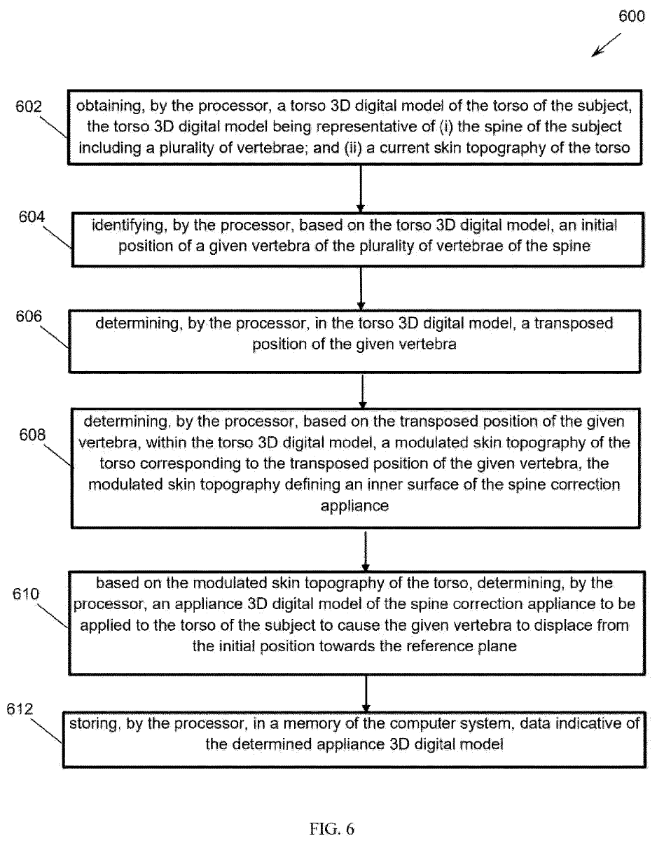

Various approaches to determining the 3D digital model of the spinal brace 200 will be described in greater detail below, with reference to FIGS. 6 to 11B and 12 to 16B, respectively.

### System

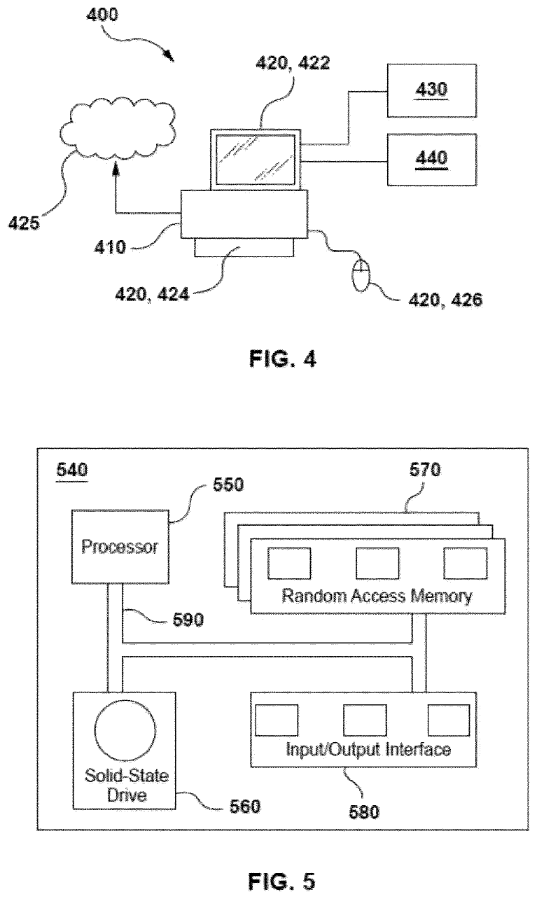

With reference to FIGS. 4 and 5, there is depicted a schematic diagram of a system 400 suitable for manufacturing of the spinal brace 200 for treating the curvature disorders of the spine 104 described above, in accordance with certain non-limiting embodiments of the present technology.

It is to be expressly understood that the system 400 as depicted is merely an illustrative implementation of the present technology. Thus, the description thereof that follows is intended to be only a description of illustrative examples of the present technology. This description is not intended to define the scope or set forth the bounds of the present technology. In some cases, what is believed to be helpful examples of modifications to the system 400 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 400 may provide in certain instances simple implementations of the present technology, and that where such is the case they have been presented in this manner as an aid to understanding. As persons skilled in the art would further understand, various implementations of the present technology may be of a greater complexity.

In certain non-limiting embodiments of the present technology, the system 400 of FIG. 4 comprises a computer system 410. The computer system 410 may be configured, by pre-stored program instructions, to determine, such as based on image data associated with the torso 102 of the subject described below, the 3D digital model of the spinal brace 200, and further cause manufacture thereof according to the 3D digital model. In other non-limiting embodiments of the present technology, the computer system can be configured to determine, based on the image data, a 3D digital of the brace mold 300 for further thermoforming the spinal brace 200.

To that end, in some non-limiting embodiments of the present technology, the computer system 410 may be configured to receive image data pertaining to the subject or to a given stage of the spine correction treatment. According to some non-limiting embodiments of the present technology, the computer system 410 may receive the image data via local input/output interface (such as USB, as an example, not separately depicted). In other non-limiting embodiments of the present technology, the computer system 410 may be configured to receive the image data over a communication network 425, to which the computer system 410 is communicatively coupled.

In some non-limiting embodiments of the present technology, the communication network 425 is the Internet and/or an Intranet. Multiple embodiments of the communication network may be envisioned and will become apparent to the person skilled in the art of the present technology. Further, how a communication link between the computer system 410 and the communication network 425 is implemented will depend, inter alia, on how the computer system 410 is implemented, and may include, but is not limited to, a wire-based communication link and a wireless communication link (such as a Wi-Fi communication network link, a 3G/4G/5G communication network link, and the like).

It should be noted that the computer system 410 can be configured for receiving the image data from a vast range of devices. Some of such devices can be used for capturing and/or processing data pertaining to a thoracis anatomy of the subject. More specifically, in certain non-limiting embodiments of the present technology, the image data received from such devices can be indicative of properties of anatomical structures of the torso 102, such as, without limitation, (i) a skeletal system of the torso 102, including the spine 104 of the subject, a ribcage, scapulae, clavicles, a sternum, a pelvis, and others; (ii) a muscle system of the torso 102 including muscles attached to the skeletal system and respective ligaments; (iii) a skin topography of the torso 102; and (iv) inner organs of the subject disposed within the torso 102, such as lungs, heart, liver, as an example.

In some non-limiting embodiments of the present technology, at least some of the image data can be indicative of properties of external portions of the anatomical structures including, for example, dimensions of each one of the plurality of vertebrae of the spine 104, such as the given vertebra 108; dimensions of respective vertebral discs of the vertebrae; and the like. Also, in some non-limiting embodiments of the present technology, the image data can be indicative of properties of the anatomical structures, including, for example, volumetric properties of the spine 104, including, for example a spinal canal receiving a spinal cord of the subject. Under certain circumstances, such volumetric properties may be factored into determining the spine correction treatment for the subject. In some non-limiting embodiments of the present technology, the image data can be intended for purposes of a particular field of medicine, such as one of orthopedics or neurosurgery, as an example.

In alternative non-limiting embodiments of the present technology, the computer system 410 may be configured to receive the image data associated with the subject directly from at least one imaging device 430 communicatively coupled thereto. Broadly speaking, a processor 550 of the computer system 410 may be configured to cause the at least one imaging device 430 to capture and/or process the image data of torso 102.

More specifically, in certain non-limiting embodiments of the present technology, the image data may include, for example, one or more of: (1) images of external surfaces of the vertebrae of the spine 104, such as the given vertebra 108 and portions thereof, such as, without limitation, a vertebral body, a vertebral foramen, pedicles, and a spinous process of the given vertebra 108, as an example; (2) images representative of the current skin topography of the torso skin 110 of the torso 102; and (3) and images of blood vessels, nerve pathways, and joint ligaments surrounding the given vertebra 108 of the spine 104. It should be noted that the image data may include two-dimensional (2D) data and/or three-dimensional data (3D). Further, in certain non-limiting embodiments of the present technology, the image data includes 2D data, from which 3D data may be derived, and vice versa.

In some non-limiting embodiments of the present technology, the at least one imaging device 430 can include an X-ray imaging system configurable to generate radiographs of the torso 102 of the subject. In some non-limiting embodiments of the present technology, the X-ray imaging system can be configured to generate a single plane radiograph of the torso 102, such as in one of the coronal or sagittal planes. However, in other non-limiting embodiments of the present technology, the X-ray imaging system can be configured to generate biplanar radiographs of the torso 102, including concurrent X-ray imaging in both the coronal and the sagittal planes of the torso 102, as an example. Also, in some non-limiting embodiments of the present technology, the X-ray imaging system can be configured to generate 3D images of the spine 104 based on these radiographs thereof.

In a specific non-limiting example, the X-ray imaging system can be of one of the types available from ATEC SPINE, Inc of 1950 Camino Vida Roble, Carlsbad, CA 92008. It should be expressly understood that the X-ray imaging system can be implemented in any other suitable equipment.

In some non-limiting embodiments of the present technology, the at least one imaging device 430 can include a magnetic resonance imaging (MRI) system. Broadly speaking, the MRI system is configured to use a phenomenon of nuclear magnetic resonance for producing 3D images of internal structures of a body of the subject, such as those of the spine 104 and of the surrounding muscles, blood vessels, nerves, and ligaments, for example.

In a specific non-limiting example, the MRI system can be of one of the MAGNETOM Sola types available from SIEMENS HEALTHCARE GMBH of Henkestr, 127 91052, Erlangen, Federal Republic of Germany. It should be expressly understood that the MRI system can be implemented in any other suitable equipment.

In yet other non-limiting embodiments of the present technology, the at least one imaging device 430 can include a computed tomography (CT) imaging system. Generally speaking, the CT imaging system comprises software and hardware allowing for capturing data using an X-ray tube rotating around the body of the subject including the torso 102. By doing so, the CT imaging system can be configured to take multiple X-ray images of the body, which can further be processed to reconstruct 3D images of the internal structures of the torso 102.

In a specific non-limiting example, the CT imaging system can be of one of the GoldSeal Optima types available from GE HEALTHCARE LTD of 3135 Easton Turnpike Chicago, IL, 06828, United States of America. It should be expressly understood that the CT imaging system can be implemented in any other suitable equipment.

In yet other non-limiting embodiments of the present technology, the at least imaging device 430 can include a 3D body scanner configured to take direct optical impressions of a surface of the body of the subject including the torso 102.

In a specific non-limiting example, the 3D body scanner can be of the Texel Portal MX type available from TEXEL LTD of Northcliffe House, Young St, London, W8 5EH, United Kingdom. It should be expressly understood that the 3D body scanner can be implemented in any other suitable equipment.

It should be expressly understood that other imaging technologies for the implementation of the at least one imaging device 430, such as an ultrasound technology, for generating the image data of the torso 102 and structures thereof, are also envisioned without departing from the scope of the present technology.

Further, it is contemplated that the computer system 410 may be configured for processing of the received image data. The resulting image data associated with the torso 102 received by the computer system 410 is typically structured as a binary file or an ASCII file, may be discretized in various ways (e.g., point clouds, polygonal meshes, pixels, voxels, implicitly defined geometric shapes), and may be formatted in a vast range of file formats (e.g., STL, OBJ, PLY, DICOM, and various software-specific, proprietary formats). Any image data file format is included within the scope of the present technology. For implementing functions described above, the computer system 410 may further comprise a corresponding computing environment.

Further, in certain non-limiting embodiments of the present technology, the system 400 may be configured to produce at least one configuration of the spinal brace 200. To that end, the system 400 can include a forming subsystem 440 configured to produce the spinal brace 200. In some non-limiting embodiments of the present technology, the forming subsystem 440 can comprise a 3D printer configured to 3D-print the spinal brace 200 according to the determined 3D digital model, as will be described in detail hereinbelow. The 3D printer may be of any suitable technology type such as one or more of: HP Jet Fusion™, selective laser sintering, polyjet, fused deposition of material (FDM), and stereolithography technologies.

In a specific non-limiting example, the 3D printer can be of one of the types of HP Jet Fusion available from HP INC. of 1501 Page Mill Road, Palo Alto, CA, 94304, United States of America. It should be expressly understood that the 3D printer can be implemented in any other suitable equipment.

However, in other non-limiting embodiments of the present technology, the forming subsystem 440 can be configured to produce the brace mold 300 for the spinal brace 200. To that end, in certain non-limiting embodiments of the present technology, the forming subsystem 440 can comprise a CNC machine configured to carve the brace mold 300 from a preform according to a 3D model representative of a desired configuration of the torso 102, determined as will be described in detail hereinbelow.

In a specific non-limiting example, the CNC machine can be of one of the types available from RODIN 4-D INC. of 27 Allée Charles Darwin, Pessac, Nouvelle-Aquitaine, 33600, France. It should be expressly understood that the CNC machine can be implemented in any other suitable equipment.

Further, as described above, by thermoforming the precursor spinal brace (not depicted) onto the brace mold 300, the spinal brace 200 can be manufactured.

Further, with reference to FIG. 5, there is depicted a schematic diagram of a computing environment 540 suitable for use with some implementations of the present technology. The computing environment 540 comprises various hardware components including one or more single or multi-core processors collectively represented by the processor 550, a solid-state drive 560, a random-access memory 570 and an input/output interface 580. Communication between the various components of the computing environment 540 may be enabled by one or more internal and/or external buses 590 (e.g., a PCI bus, universal serial bus, IEEE 1394 “Firewire” bus, SCSI bus, Serial-ATA bus, ARINC bus, etc.), to which the various hardware components are electronically coupled.

The input/output interface 580 allows enabling networking capabilities such as wire or wireless access. As an example, the input/output interface 580 comprises a networking interface such as, but not limited to, a network port, a network socket, a network interface controller and the like. Multiple examples of how the networking interface may be implemented will become apparent to the person skilled in the art of the present technology. For example, but without being limiting, the input/output interface 580 may implement specific physical layer and data link layer standard such as Ethernet™, Fibre Channel, Wi-Fi™ or Token Ring™. The specific physical layer and the data link layer may provide a base for a full network protocol stack, allowing communication among small groups of computers on the same local area network (LAN) and large-scale network communications through routable protocols, such as IP.

According to implementations of the present technology, the solid-state drive 560 stores program instructions suitable for being loaded into the random-access memory 570 and executed by the processor 550, according to certain aspects and embodiments of the present technology. For example, the program instructions may be part of a library or an application.

In some non-limiting embodiments of the present technology, the computing environment 540 is implemented in a generic computer system, which is a conventional computer (that is, an “off the shelf” generic computer system). The generic computer system may be a desktop computer/personal computer, but may also be any other type of electronic device such as, but not limited to, a laptop, a mobile device, a smart phone, a tablet device, or a server.

As persons skilled in the art of the present technology may appreciate, multiple variations as to how the computing environment 540 can be implemented may be envisioned without departing from the scope of the present technology.

Referring back to FIG. 4, the computer system 410 has at least one interface device 420 for providing an input or an output to a user of the system 400, the interface device 420 being in communication with the input/output interface 580. In the embodiment of FIG. 4, the interface device is a screen 422. In other non-limiting embodiments of the present technology, the interface device 420 may be a monitor, a speaker, a printer, or any other device for providing an output in any form such as an image form, a written form, a printed form, a verbal form, a 3D model form, or the like.

In the depicted embodiments of FIG. 4, the interface device 420 also comprises a keyboard 424 and a mouse 426 for receiving input from the user of the system 400. Other interface devices 420 for providing an input to the computer system 410 can include, without limitation, a USB port, a microphone, a camera, or the like.

The computer system 410 may be connected to other users, such as through their respective clinics, through a server (not depicted). The computer system 410 may also be connected to stock management or client software which could be updated with stock when the orthodontic treatment has been determined and/or schedule appointments or follow-ups with clients, for example.

Thus, given the architecture and examples provided above, it is now possible to execute the present methods for determining the 3D model of the spinal brace 200, in accordance with certain non-limiting of the present technology. According to non-limiting embodiments of the present technology, any of the methods described herein can be executed by the processor 550 of the computing environment 540.

Furthermore, the methods and systems described herein include the following approaches to determining the 3D digital model. One approach, referred to herein as a “mirroring” approach, is directed to determining a configuration of the torso 102 including a transposed position of the given vertebra 108 within the more detailed 3D digital model, which can be determined by mirroring a current position of the given vertebra 108 relative to at least one of the sagittal, coronal, and transverse plane associated with the subject, as depicted in FIG. 1A.

Another approach, referred to herein as an “optimization” approach, includes optimizing a raw configuration of the spinal brace 200 determined, for example, by the mirroring approach, by considering the parameters indicative of safety and wear comfort of the spinal brace 200 as well as growth-related changes of the spine 104 during the spine correction treatment. By doing so, the present methods may allow determining an optimized configuration of the spinal brace 200 which may help increase the biomechanical efficacy of the spine correction treatment over time and the subject's adherence thereto, thereby increasing the overall effectiveness of the spine correction treatment.

Combinations of aspects of the first and second approaches are also within the scope of the present technology.

### Mirroring Approach

With reference to FIG. 6, there is depicted a schematic diagram of a method 600 for determining a model of the spine correction appliance, such as the spinal brace 200 described above, in accordance with certain non-limiting embodiments of the present technology.

Step 602: Obtaining, by the Processor, a Torso 3D Digital Model of the Torso of the Subject, the Torso 3D Digital Model being Representative of (I) the Spine of the Subject Including a Plurality of Vertebrae; and (II) a Current Skin Topography of the Torso

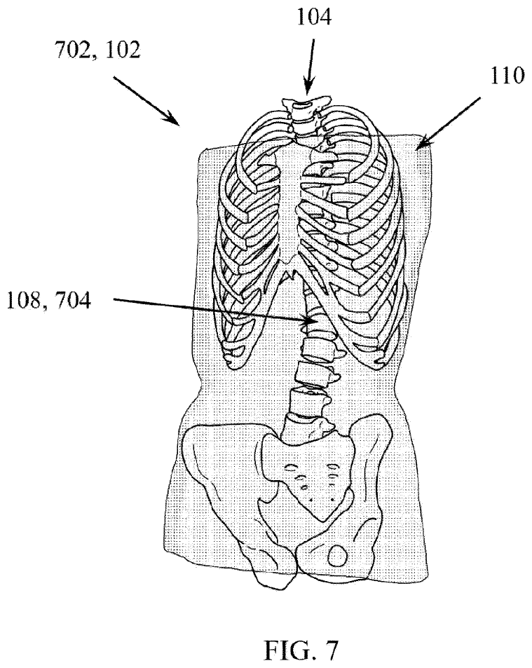

The method 600 commences at step 602 with the processor 550 being configured to obtain a torso 3D digital model 702 of the torso 102 of the subject, depicted in FIG. 7, in accordance with certain non-limiting embodiments of the present technology.

According to certain non-limiting embodiments of the present technology, the torso 3D digital model 702 can include (i) a skeleton 3D digital model (not separately labelled) of the skeletal system of the torso 102 including the spine 104; and (ii) a skin 3D digital model (not separately labelled) of a current skin topography of the torso skin 110 of the torso 102, as depicted in FIG. 7. In the depicted embodiments of the torso 3D digital model 702, aside from the digital representation of the spine 104, the skeletal system also includes the ribcage and the pelvis of the subject. However, in other non-limiting embodiments of the present technology, the torso 3D digital model 702 can also be representative of fewer or more structures of the skeletal system of the torso 102. In one example, the torso 3D digital model 702 can further be representative of other structures of the skeletal system of the torso 102, such as clavicles, scapulae, a sternum, and the like. In yet other non-limiting embodiments of the present technology, the torso 3D digital model 702 can be representative of the skeletal system including only the spine 104. In yet other non-limiting embodiments of the present technology, the torso 3D digital model 702 can include a muscle 3D digital model (not depicted) of the muscle system of the subject including muscles and ligaments attached to the skeletal system.

In some non-limiting embodiments of the present technology, the torso 3D model 702 can comprise a plurality of mesh elements representative of surfaces of each one of the above-mentioned anatomical structures. A shape of a given mesh element of the plurality of mesh elements is not limited, and the given mesh element can be, for example, a triangular mesh element. However, it should be expressly understood that in other non-limiting embodiments of the present technology, the plurality of mesh elements may include quadrilateral mesh elements, convex polygonal mesh elements, or even concave polygonal mesh elements, as an example, without departing from the scope of the present technology.

In some non-limiting embodiments of the present technology, the torso 3D digital model 702 can be configured to display (such as in real time) modifications to the skin 3D digital model of the torso skin 110 in response to displacing at least one of the vertebrae of the spine 104 within the skeleton 3D digital model, such as the given vertebra 108. In this regard, in some non-limiting embodiments of the present technology, the torso 3D digital model 702 can be implemented as a finite element model (FEM) representative of biomechanical properties, such as elasticity, for example, of the above-mentioned anatomical structures of the torso 102 under a given force.

It is not limited how the processor 550 can be configured to obtain the torso 3D digital model 702. In one example, the torso 3D digital model 702 can be generated, based on the image data mentioned above, by a third-party finite element analysis (FEA) software and stored in a format receivable by the processor 550. Such a software can be configured to conduct a FEA for various objects, such as the torso 102 and internal structures thereof, that is, solving differential equations, describing a behavior of the torso 102 and structures thereof under the given force, using the finite element method to determine, for each mesh element of the plurality of mesh elements defining surfaces of the torso 3D digital model 702, a respective modulus of elasticity.

In a specific non-limiting example, the FEA software can be of one of the types available from ANSYS INC. of Southpointe 2600 Ansys Drive Canonsburg, PA 15317, United States of America. It should be expressly understood that the 3D printer can be implemented in any other suitable equipment.

However, in other non-limiting embodiments of the present technology, the processor 550 can be configured to generate the torso 3D digital model 702. More specifically, the processor 550 can be configured to (1) receive the image data of torso 102, including that of the skeletal system and the torso skin 110, (2) generate the skeletal 3D digital model and the skin 3D digital model of the torso 102; (3) merge the skeletal 3D digital model and the skin 3D digital model; and optionally (4) conduct the FEA of the so merged digital models.

According to certain non-limiting embodiments of the present technology, to generate the skeleton 3D digital model of the skeletal system of the torso 102, the processor 550 can be configured to receive, such as from the at least one imaging device 430 described above, biplanar radiographs (not depicted) of the skeletal system representative of a configuration of the skeletal system in the coronal and the sagittal planes associated with the subject. Further, the processor 550 can be configured to reconstruct, based on the biplanar radiographs, the skeleton 3D digital model of the skeletal system of the subject. To that end, the processor 550 can be configured to register certain predetermined reference points on both radiographs such that bony elements, for example, represented by the resultant skeleton 3D digital model of the torso 3D digital model 702 would correspond to their representations in each one of the biplanar radiographs.

Further, according to certain non-limiting embodiments of the present technology, the processor 550 can be configured to generate the skin 3D digital model based on a 3D scan, also received from the at least one imaging device 430 described above, of the surface of the torso 102 including mesh elements representative of the current skin topography of the torso skin 110.

Further, once the processor 550 has generated each one of the skeleton 3D digital model and the skin 3D digital model of the torso 102, the processor 550 can further be configured to merge them in a merged 3D digital model. To that end, the processor 550 can be configured to register, within the merged 3D digital model, positions of certain reference objects relative to the torso skin 110 identified in the biplanar radiographs. For example, in some non-limiting embodiments of the present technology, such reference objects can include certain vertebrae, such as T1 and L5 vertebrae (not separately labelled) of the plurality of vertebrae of the subject. In other non-limiting embodiments of the present technology, such reference objects can include the pelvis or ribcage, or sternum, as an example.

Further, the processor 550 can be configured to execute the FEA software to conduct the FEA for the merged 3D digital model including modelling application of gravity forces and forces from the spinal brace 200 onto the torso 102, thereby determining the torso 3D digital model 702 thereof. Thus, by doing so, the processor 550 can be configured to obtain the torso 3D digital model 702 where the skin 3D digital model (not separately labelled) is responsive to changes of the skeleton 3D digital model (not separately labelled), such as displacements of at least one of the plurality of vertebrae forming the spine 104, such as the given vertebra 108.

Also, as it can be appreciated, in different positions of the torso 102, stresses caused by gravity forces and the forces from the spinal brace 200 are distributed along the spine 104 differently. To that end, different configurations of the spinal brace 200 may be required, for example, for a time when the subject is in a standing position (also referred to herein as a “full-time spinal brace”) and when the subject is in a lying position, such as during the night-time (also referred to herein as a “night-time spinal brace”). The lying position can include, without limitation, at least one of a prone position, a supine position, a left and right lateral recumbent positions, a Fowler's position, and a Trendelenburg's position, and others.

Thus, in some non-limiting embodiments of the present technology, the processor 550 can be configured to determine different configurations of the torso 3D digital model 702 of the torso 102 for the different positions of the subject, based on which the different respective configurations of the spinal brace 200 may further be manufactured, as will be described in detail below. Accordingly, in these embodiments, the processor 550 can be configured to conduct the FEA for the merged 3D digital model modelling the application of the gravity forces and the forces from the spinal brace 200 onto the torso 102 to simulate the respective different stress distributions along the spine 104. Additionally, to simulate the stress distribution along the spine 104 while the subject is in one of the above-listed lying positions, the processor 550 is configured to use a pre-determined surface 3D digital model (also generated using the FEA, as an example, not depicted) of a surface on which the subject is to spend their nighttime during the spine correction treatment with the spinal brace 200. For example, such a surface 3D digital model can be configured to model a surface of a mattress on which the subject normally sleeps.

As mentioned above, it is not limited which approach the processor 550 can be configured to apply to the merged 3D digital model for conducting the FEA thereof to generate the torso 3D digital model 702; however, in specific non-limiting embodiments of the present technology, the processor 550 can be configured to apply one of the approaches described in an article entitled “A New Method to Include The Gravitational Forces in a Finite Element Model of the Scoliotic Spine”, authored by Clin et al. and published by International Federation for Medical and Biological Engineering on Jul. 5, 2011, the content of which is incorporated herein by reference in its entirety.

The method 600 hence advances to step 604.

### Step 604: Identifying, by the Processor, Based on the Torso 3D Digital Model, an Initial Position of a Given Vertebra of the Plurality of Vertebrae of the Spine

At step 604, according to certain non-limiting embodiments of the present technology, the processor 550 can be configured to identify, using the torso 3D digital model 702 obtained at step 602, an initial (or otherwise current) position of each one of the plurality of vertebrae forming the spine 104 within the torso 102, such as a respective initial position 704 of the given vertebra 108.

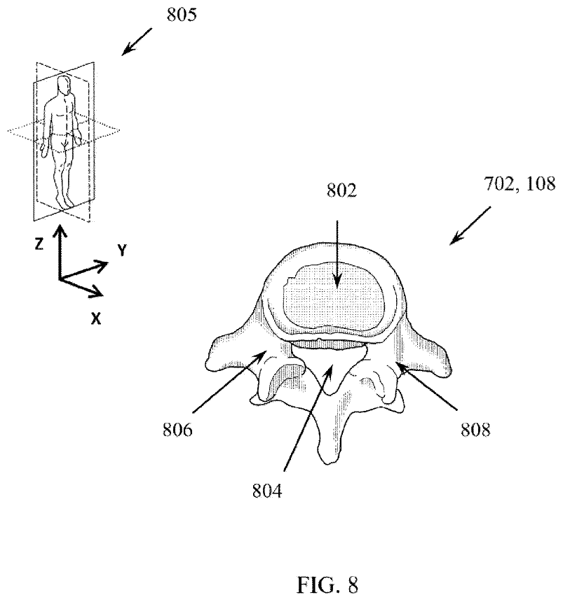

With continued reference to FIG. 7 and with reference to FIG. 8, there is depicted a schematic diagram of anatomical features of the given vertebra 108 of the plurality of vertebrae of the spine 104 as represented by the torso 3D digital model 702, in accordance with certain non-limiting embodiments of the present technology.

In some non-limiting embodiments of the present technology, to identify the initial position of the given vertebra 108 within the torso 102 using the torso 3D digital model 702, first, the processor 550 can be configured to define, around the torso 3D digital model a reference coordinate system 805. For example, in some non-limiting embodiments of the present technology, the processor 550 can be configured to define the reference coordinate system 805 to be formed by anatomical planes associated with the subject, that is, the sagittal, coronal, and the transverse planes. It is not limited how the processor 550 can be configured to obtained data of the anatomical planes; and in some non-limiting embodiments of the present technology, the processor 550 can be configured to determine the anatomical planes based on considerations of bilateral symmetry of the subject's body. More specifically, the processor 550 can be configured to determine the sagittal plane as extending, in the torso 3D digital model 702, through midlines of structures of the subject's body, such as through the spine 104, and more specifically, through a centroid of an upper plate of a reference vertebra (such as L5, for example, not separately labelled); a navel (not depicted); through certain muscle groups, such as abdominal muscles (not depicted); and the like. Further, the processor 550 can be configured to determine the coronal plane as extending, in the torso 3D digital model 702, as a plane perpendicular to the sagittal plane dividing the torso 102 into dorsal and ventral (back and front) portions. Finally, the processor 550 can be configured to determine the transverse plane as being perpendicular to both the sagittal and the coronal planes.

Further, according to certain non-limiting embodiments of the present technology, the processor 550 can be configured to determine the respective initial position 704 of the given vertebra 108 in the torso 3D digital model 702 by determining coordinates, in the reference coordinate system 805, of one or more reference points of the given vertebra 108.

It is not limited how a given reference point of the given vertebra 108 can be determined. In some non-limiting embodiments of the present technology, the given reference point can be determined as a center mass point of a vertebral body 802 of the given vertebra 108. In other non-limiting embodiments of the present technology, the given reference point can be determined as a center of a vertebral foramen 804 of the given vertebra 108, defining the spinal canal of the spine 104. In yet other non-limiting embodiments of the present technology, the given reference point can be representative of a center point of at least one of a left pedicle 806 and a right pedicle 808 (in the orientation of FIG. 8) of the given vertebra 108. Other manners of determining the given reference point, such as a being representative of a spinous or one of transverse processes (not separately labelled) of the given vertebra 108, are also envisioned without departing from the scope of the present technology.

The method 600 hence proceeds to step 606.

### Step 606: Determining, by the Processor, in the Torso 3D Digital Model, a Transposed Position of the Given Vertebra

At step 606, according to certain non-limiting embodiments of the present technology, the processor 550 can be configured to obtain an indication of a respective target position of the given vertebra 108, towards which the given vertebra 108 of the plurality of vertebrae of the spine 104 is to be caused to displace by applying the spinal brace 200 manufactured as described herein. For example, as mentioned above, the processor 550 can be configured to determine the respective target position of the give vertebra 108 as corresponding to the normal curvature of the spine 104, schematically depicted in FIG. 1B.

Further, to determine a configuration of the spinal brace 200 causing the given vertebra 108 to move towards the target position thereof, in some non-limiting embodiments of the present technology, first, the processor 550 can be configured to determine a transposed position for the given vertebra 108. According to certain non-limiting embodiments of the present technology, the transposed position of the given vertebra is indicative of an overcorrected position of the given vertebra 108 within the spine 104. Thus, the processor 550 can be configured to determine the transposed position for the given vertebra 108 such that the respective target position thereof would be between the initial position (such as the respective initial position 704) and the transposed position of the given vertebra 108.

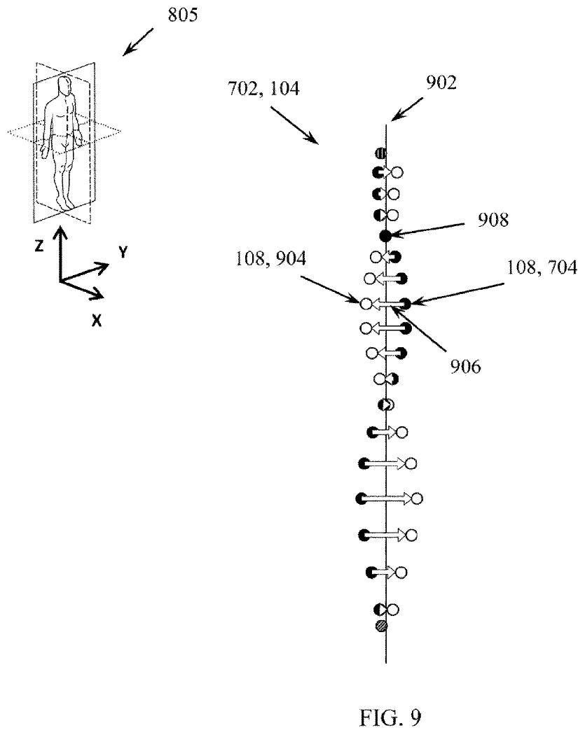

With reference to FIG. 9, there is depicted a schematic diagram for determining, within the torso 3D digital model 702, a respective transposed position 904 of the given vertebra 108, in accordance with certain non-limiting embodiments of the present technology.

According to certain non-limiting embodiments of the present technology, the processor 550 can be configured to determine the respective transposed position 904 of the given vertebra 108 by mirroring the respective initial position 704 thereof, identified at step 604, relative to a reference plane 902 associated with the subject. As mentioned above with reference to FIG. 8, the respective initial position 704 can be represented by the center mass point of the vertebral body 802 of the given vertebra 108, as an example.

According to certain non-limiting embodiments of the present technology, the reference plane 902 can be determined as corresponding to the respective target position of the given vertebra 108. Thus, in those embodiments where the curvature disorder of the spine 104 is in the coronal plane (also known as “scoliosis”), such as that of the given vertebra 108, the processor 550 can be configured to determine the reference plane 902 as being a symmetry plane associated with the spine 104, intersecting the coronal plane, such as the sagittal plane associated with the subject. To that end, the processor 550 can be configured to determine the respective transposed position 904 of the given vertebra 108 as a mirrored position of the respective initial position 704 thereof relative to the reference plane 902 within the coronal plane associated with the subject, as depicted in FIG. 9.

Also, in additional non-limiting embodiments of the present technology (not depicted), the processor 550 can be configured to determine the respective transposed position 904 of the given vertebra 108 as a mirrored position of the respective initial position 704 not only in the coronal plane, but also in the transverse plane associated with the subject.

However, in those embodiments (not depicted), where the curvature disorder of the spine 104 also occurs in the transverse plane associated with the subject, that is, when at least some of the plurality of vertebrae of the spine 104 have been axially rotated relative to the coronal plane around their longitudinal axes, the processor 550 can be configured to determine the reference plane 902 as being the sagittal plane rotated relative to the coronal plane until the sagittal plane encompasses a maximum curvature of the spine 104 in the transverse plane.

Further, as mentioned hereinabove, based on the respective transposed position 904 of the given vertebra 108, the respective target position thereof can be determined. In some non-limiting embodiments of the present technology, the respective target position can be determined as corresponding to a midpoint of a line segment extending between the respective initial position 704 and the respective transposed position 904—such as a midpoint (not separately labelled) of a coronal line segment 906 extending between the respective initial position 704 and the respective transposed position 904 in the coronal plane as depicted in FIG. 9, as an example.

However, in other non-limiting embodiments of the present technology, the spine correction treatment for moving the given vertebra 108 from the respective initial position 704 to the respective target position thereof can be divided into a plurality of stages, during which a respective configuration of the spinal brace 200 can be applied. To that end, the processor 550 can be configured to determine respective intermediate transposed positions of the given vertebra 108 for each of the plurality of stages. In some non-limiting embodiments of the present technology, a given one of the respective intermediate transposed positions of the given vertebra 108 can be determined as the mirrored position with a respective value of a weight coefficient. The weight coefficient can take values from 0 to 1, where 0 corresponds to a zero mirroring of the respective initial position 704, that is, when the given intermediate transposed position is a level of the reference plane 902; and 1 corresponds to a full mirroring of the respective initial position 704, such as that depicted in FIG. 9.

In other words, the processor 550 can be configured to determine the given one of the respective intermediate transposed positions for the given vertebra 108 at a distance, from the reference plane 902, corresponding to the full mirroring of the respective initial position 704 taken with the respective value of the weight coefficient. For example, if the number of stages has been determined as five, respective values of the weight coefficient can be determined with predetermined step, such as 0.2: 0.2, 0.4, 0.6, 0.8, and 1. Determining the respective values for the weight coefficient with different steps at each of the stage of the spine correction treatment is also envisioned. Accordingly, for each of the stages, the processor 550 can further be configured to determine a respective intermediate target position for the given vertebra 108, as described above.

In yet other non-limiting embodiments of the present technology, when determining the respective transposed position 904 of the given vertebra 108, the processor 550 can be configured to consider the misalignment thereof not only within a single plane, such as at least one of the coronal plane and the transverse plane, as described above, but in three dimensions, within the reference coordinate system 805.

To that end, the processor 550 can be configured to determine coordinates of the respective initial position 704 of the given vertebra 108 and further, based thereon, determine coordinates of the respective transposed position 904 within the reference coordinate system 805. In these embodiments, to consider rotation of the given vertebra 108 within the transverse plane, the processor 550 can be configured to determine the respective initial position 704 of the given vertebra 108 as initial coordinates of the right pedicle 808 of the given vertebra 108 in the reference coordinate system 805.

Further, according to certain non-limiting embodiments of the present technology, the processor 550 can be configured to determine transposed coordinates of each one of the right and left pedicles 806, 808 of the given vertebra 108, thereby determining the respective transposed position 904 thereof within the reference coordinate system 805. In some non-limiting embodiments of the present technology, the processor 550 can be configured to determine the transposed coordinates for each one of the left and left pedicles 806, 808, by applying displacements thereto on the reference coordinate system 805, the displacements being determined in accordance with following equations:

$${u}_{x L} = Wx * ( {x}_{R} - {x}_{L} ) ; \qquad ( 1 )$$

$${u}_{x R} = Wx * ( {x}_{L} - {x}_{R} ) ;$$

$${u}_{y L} = - Wy * ( {y}_{R} + {y}_{L} ) ;$$

$${u}_{y R} = - Wy * ( {y}_{L} + {y}_{R} ) ;$$

$${u}_{zL} = Wz * ( {z}_{R} - {z}_{L} ) ; and$$

$${u}_{zR} = Wz * ( {z}_{L} - {z}_{R} ) ;$$

where x_{R} is an X coordinate of the right pedicle in respective initial position 704;
   x_{L} is an X coordinate of the left pedicle in respective initial position 704;
   u_{xL} is an X displacement applied to the X coordinate of the left pedicle to obtain an X coordinate thereof in respective transposed position 904 of the given vertebra, x_{L}+u_{xL};
   u_{xR} is an X displacement applied to the X coordinate of the right pedicle to obtain an X coordinate thereof in respective transposed position 904 of the given vertebra, x_{R}+u_{xR};
   y_{R} is a Y coordinate of the right pedicle in respective initial position 704;
   y_{L} is a Y coordinate of the left pedicle in respective initial position 704;
   u_{yL} is a Y displacement applied to the Y coordinate of the left pedicle to obtain a Y coordinate thereof in respective transposed position 904 of the given vertebra, y_{L}+
   u_{yL};
   u_{yR} is a Y displacement applied to the Y coordinate of the right pedicle to obtain a Y coordinate thereof in respective transposed position 904 of the given vertebra, y_{R}+
   u_{yR};
   z_{R} is a Z coordinate of the right pedicle in respective initial position 704;
   Z_{L} is a Z coordinate of the left pedicle in respective initial position 704;
   U_{zL} is a Z displacement applied to the Z coordinate of the left pedicle to obtain a Z coordinate thereof in respective transposed position 904 the given vertebra, z_{L}+u_{zL}
   u_{zR} is a Z displacement applied to the Z coordinate of the right pedicle to obtain a Z coordinate thereof in respective transposed position 904 of the given vertebra, z_{R}+U_{zR}
Wx, Wy, and Wz are respective weight coefficients, determined, for each one of the coordinates of the reference coordinate system 805, like the weight coefficient described above.

In this regard, the processor 550 can be configured to determine the target position of the given vertebra 108 in the reference coordinate system 805 as respective target coordinates of the left and right pedicle 806, 808 that are determined by applying target displaces to the coordinates of the left and right pedicles 806, 806 in the respective initial position 704 of the given vertebra 108. According to certain non-limiting embodiments of the present technology, the processor 550 can be configured to determine the target displacements according to following equations:

$${u}_{xL}^{T} = 0.5 * {u}_{x L} ; \qquad ( 2 )$$

$${u}_{xR}^{T} = 0.5 * {u}_{x R} ;$$

$${u}_{yL}^{T} = - 0.5 * {u}_{yL} ;$$

$${u}_{yR}^{T} = - 0.5 * {u}_{yR} ;$$

$${u}_{zL}^{T} = 0.5 * {u}_{zL} ; and$$

$${u}_{zR}^{T} = 0.5 * {u}_{z R} .$$

According to certain non-limiting embodiments of the present technology, the processor 550 can be configured to determine the initial and transposed coordinates of the left and right pedicles 806, 808 as coordinates of first and second reference points defined therewithin, respectively. For example, the first and second reference points can be determined as center mass points of the left and right pedicles 806, 808.

By doing so, the processor 550 can be configured to determine respective transposed positions for each one of the plurality of vertebrae forming the spine 104, as described above with respect to the given vertebra 108. However, in other non-limiting embodiments of the present technology, first, the processor 550 can be configured to (i) determine, in the torso 3D digital model 702, based on the respective initial position 704, if the given vertebra 108 is misaligned; and (ii) determine the respective transposed position 904 only if the given vertebra 108 is misaligned.

In other words, the processor 550 can be configured not to determine respective transposed positions for those of the plurality of vertebrae of the spine 104 that are considered aligned therewithin-such as another vertebra 908 depicted in FIG. 9. It is not limited how the processor 550 can be determined if the given vertebra 108 is misaligned, and can include determining if a deviation distance of the given vertebra 108 (such as the given reference point thereof described above) from at least one of the sagittal, coronal, and transverse planes is shorter than a predetermined distance threshold, such as 0.5 mm, 1.0 mm, or 1.5 mm, as an example.

The method 600 hence advances to step 608.

Step 608: Determining, by the Processor, Based on the Transposed Position of the Given Vertebra, within the Torso 3D Digital Model, a Modulated Skin Topography of the Torso Corresponding to the Transposed Position of the Given Vertebra, the Modulated Skin Topography Defining an Inner Surface of the Spine Correction Appliance

At step 608, based on the respective transposed position of at least one of the plurality of vertebrae of the spine 104, such as the respective transposed position 904 of the given vertebra 108, according to certain non-limiting embodiments of the present technology, the processor 550 can be configured to determine, within the torso 3D digital model 702, a modulated skin topography of the torso skin 110 of the torso 102. To that end, the processor 550 can be configured to cause, within the torso 3D digital model 702, displacement of the given vertebra 108 from the respective initial position 704 to the respective transposed position 904 thereof.

As mentioned hereinabove, in the embodiments where the torso 3D digital model 702 is a FEM, to cause the displacement of the given vertebra 108 therein, the processor 550 can be configured to simulate application of a force on the given vertebra 108 causing it to displace to the respective transposed position 904. For example, the processor 550 can be configured to simulate application of spring elements to the given vertebra 108 having respective elastic moduli predetermined to cause the given vertebra 108 to move towards the respective transposed position 904 thereof. In another example, the processor 550 can be configured to simulate, in the torso 3D digital model 702, application of a torsional moment to the given vertebra 108 causing the given vertebra 108 to move towards the respective transposed position 904. Other types of forces that can be applied to the given vertebra 108, in the torso 3D digital model 702, causing the given vertebra 108 to move to the respective transposed position 904 are also envisioned.

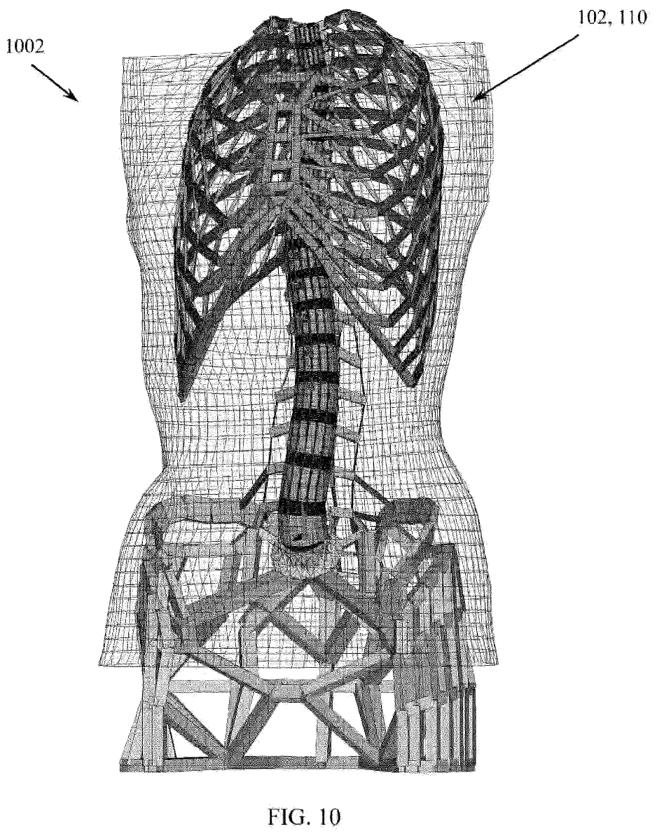

With reference to FIG. 10, there is depicted a schematic diagram of a modified torso 3D digital model 1002 generated, by the processor 550, in response to displacing, within the torso 3D digital model 702, the given vertebra 108 from the respective initial position 704 to the respective transposed position 904 thereof, in accordance with certain non-limiting embodiments of the present technology.

As mentioned hereinabove, the torso 3D digital model 702 is configured to reflect changes in the skin 3D digital model of the torso skin 110 in response to the changes in the skeleton 3D digital model, such as displacing the given vertebra 108 of the plurality of vertebrae from the respective initial position 704 to the respective transposed position 904 thereof. Thus, the modified torso 3D digital model 1002 is different from the torso 3D digital model 702 depicted in FIG. 7 in that the former is indicative of the modulated skin topography resulting from the displacement of the given vertebra 108 from the respective initial position 704 to the respective transposed position 904.

As can further be appreciated, in those embodiments where at step 606 the processor 550 is configured to determine the respective transposed positions for each one of the plurality of vertebrae of the spine, the processor 550 can further be configured to determine the modified 3D digital model 1002 indicative of the modulated skin topography of the torso skin 110 resulting from displacement of each one of the plurality of vertebrae to their respective transposed positions.

Also, in some non-limiting embodiments of the present technology, the processor 550 can be configured to determine the modified 3D digital model 1002 factoring in growth-related changes of the skeletal system of the subject during the spine correction treatment, including at least changes of the spine 104. To do so, according to certain non-limiting embodiments of the present technology, the processor 550 can be configured to (i) obtain the biometrical reference data of the subject, indicative of how structures of the skeletal system thereof, such as the plurality of vertebrae of the spine 104, change in size over a treatment period of the spine correction treatment; (ii) modify, based on the obtained reference data, using the obtained reference data, the torso 3D digital model 702, to determine an updated configuration (not depicted) of the spine 104 by a moment of interest during the treatment period, such as an end thereof; and (iii) cause the so modified configuration of the given vertebra 108 of the plurality of vertebrae to move, in the torso 3D digital model 702, to the respective transposed position 904, thereby determining the modified 3D digital model 1002 including the updated configuration of the spine 104.

Thus, the processor 550 can be configured to determine the modified torso 3D digital model 1002 whose outer surface defines an inner surface of the spinal brace 200.

The method 600 hence advances to step 610.

Step 610: Based on the Modulated Skin Topography of the Torso, Determining, by the Processor, an Appliance 3D Digital Model of the Spine Correction Appliance to be Applied to the Torso of the Subject to Cause the Given Vertebra to Displace from the Initial Position Towards the Reference Plane

At step 610, according to certain non-limiting embodiments of the present technology, the processor 550 can be configured to determine, based on the modulated skin topography of the torso skin 110 indicated by the modified torso 3D digital model 1002, the 3D digital model of the spinal brace 200.

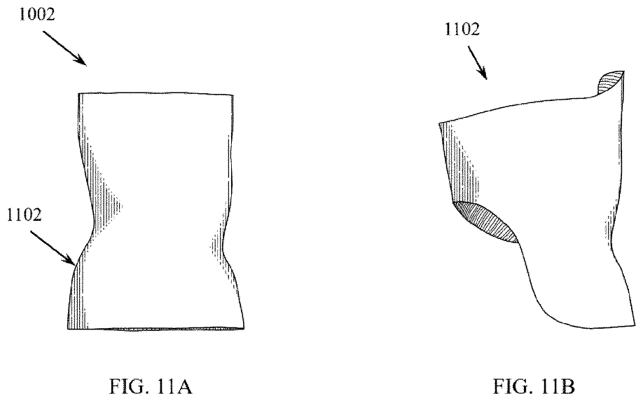

With reference to FIGS. 11A and 11B, there is depicted a schematic diagram of a brace 3D digital model 1102 of the spinal brace 200 generated, by the processor 550, based on the modified torso 3D digital model 1002, in accordance with certain non-limiting embodiments of the present technology.

According to certain non-limiting embodiments of the present technology, to generate the brace 3D digital model 1102, the processor 550 can be configured to: (1) receive a precursor brace 3D digital model (not depicted) which, akin to other 3D digital models described above, in some non-limiting embodiments of the present technology, can be determined based on an FEA and comprise mesh elements defining a surface thereof; (2) model stretching, under a predetermined simulated force, the precursor brace 3D digital model around the modified torso 3D digital model 1002, thereby forming an inner surface of the brace 3D digital model 1102. Accordingly, the inner surface of the brace 3D digital model 1102 defines the inner surface 204 of the spinal brace 200, in certain embodiments.

The method 600 thus proceeds to step 612.

### Step 612: Storing, by the Processor, in a Memory of the Computer System, Data Indicative of the Determined Appliance 3D Digital Model

At step 612, the processor 550 can be configured to store the brace 3D digital model 1102 determined at step 610 in a memory of the computing environment 540, such as in the solid-state drive 560, for further use in development the spine correction treatment, as an example.

In some non-limiting embodiments of the present technology, the processor 550 can be configured to cause display, such as on the screen 422 of the system 400, of the brace 3D digital model 1102 for presentation thereof, for example, to an orthopedic practitioner or the subject.

Alternatively or additionally, in some non-limiting embodiments of the present technology, at step 612, the processor 550 can be configured to cause manufacture of the spinal brace 200 according to the brace 3D digital model 1102. To that end, as described above with reference to FIG. 4, the processor 550 can be configured to use the forming subsystem 440. More specifically, in those embodiments where the forming subsystem 440 comprises the 3D printer, the processor 550 can be configured to cause direct 3D-printing of the spinal brace 200 according to the brace 3D digital model 1102. In those embodiments where the forming subsystem 440 comprises the CNC machine, the processor 550 can be configured to cause the forming subsystem 440 to manufacture the brace mold 300 for further thermoforming thereon the spinal brace 200.

As mentioned hereinabove, the method 600 can include a single iteration for determining the brace 3D digital model 1102 for the configuration of the spinal 200 causing the given vertebra 108 to move to the respective target position in one step. However, in those embodiments where the spine correction treatment includes the plurality of stages, the processor 550 can be configured to run the method 600 a respective number of times to determine brace 3D digital models for the respective configurations of the spinal brace 200 progressively causing the given vertebra 108 to move towards the respective target position in multiple steps.

The method 600 thus terminates.

Thus, certain non-limiting embodiments of the method 600 allow increasing efficiency of manufacturing the spinal brace 200 due to the specific approach to determining a configuration thereof, based on the respective transposed positions of the plurality of vertebrae of the spine 104.

Further, certain non-limiting embodiments of the present technology are directed to a method of optimizing a given configuration of the spinal brace 200 considering parameters indicative of the safety of and wear conform thereof, as well as the growth-related changes of the spine 104 of the subject during the spine correction treatment.

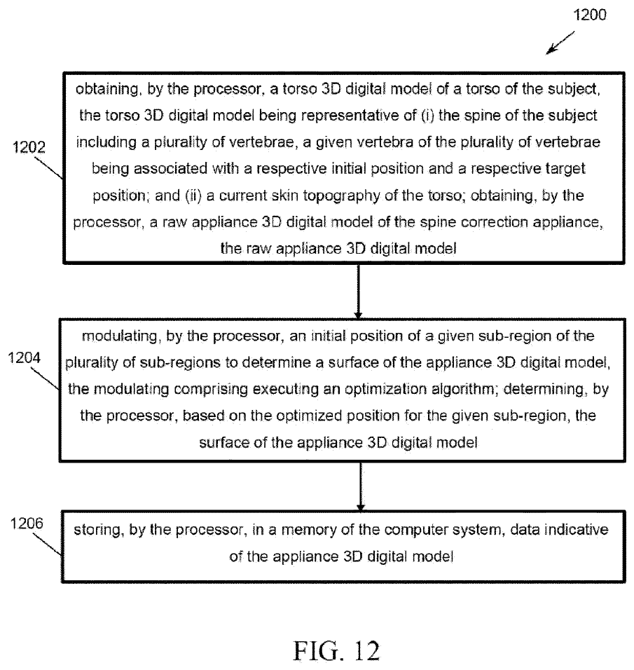

With reference to FIG. 12, there is depicted a flowchart diagram of another method 1200 of determining the 3D digital model of a spine correction appliance, such as that of the spinal brace 200, in accordance with certain non-limiting embodiments of the present technology. Akin to the method 600, the method 1200 can also be executed by the processor 550 of the computing environment 540.

Step 1202: Obtaining, by the Processor, a Torso 3D Digital Model of a Torso of the Subject, the Torso 3D Digital Model being Representative of (I) the Spine of the Subject Including a Plurality of Vertebrae, a Given Vertebra of the Plurality of Vertebrae being Associated with a Respective Initial Position and a Respective Target Position; and (II) a Current Skin Topography of the Torso; Obtaining, by the Processor, a Raw Appliance 3D Digital Model of the Spine Correction Appliance, the Raw Appliance 3D Digital Model

According to certain non-limiting embodiments of the present technology, the method 1200 is directed to determining the 3D digital model of the spinal brace 200 by optimizing a raw 3D digital model thereof. Thus, at step 1202, according to certain non-limiting embodiments of the present technology, the processor 550 can be configured to obtain input image data. More specifically, the processor 550 can be configured to obtain a 3D digital model of the torso 102 of the subject, such as the torso 3D digital model 702, described above, combining the 3D digital models of the skeletal system and the current skin topography of the torso skin 110 of the subject. Further, in some non-limiting embodiments of the present technology, the processor 550 can be configured to obtain a raw brace 3D digital model of the spinal brace 200, based on which, the processor 550 can be configured to determine an optimized configuration of the spinal 200 executing further steps the method 1200.

In the context of the present specification, the raw brace 3D digital model of the spinal brace 200 is indicative of a raw configuration of the spinal brace 200, which when worn onto the torso 102 of the subject, causes at least one misaligned vertebra of the spine 104, such as the given vertebra 108 of the misaligned chain 106, to move to its respective target position. However, the raw configuration of the spinal brace 200 does not consider safety, wear comfort, or the growth of the subject during the spine correction treatment. By way of example, the raw configuration of the spinal brace 200 can be configured to cause the given vertebra 108 to move to its respective target position in a shorter time, but may cause excess pressure on at least one of the anatomical structures of the subject, such as the torso skin 110, the spine 104, or the inner organs, which would result in discomfort to the subject, or even an injury, during the implementation of the spine correction treatment.

It is not limited how the raw brace 3D digital model of the spinal brace 200 can be obtained, and in some non-limiting embodiments of the present technology, the raw brace 3D digital model can be generated by a third-party software and stored in a format receivable by the processor 550. In other non-limiting embodiments of the present technology, a raw configuration of the spinal brace 200 can be determined manually, such as by the orthopedic practitioner; and the processor 550 can be configured to receive 3D impressions thereof, for example, from the imaging device 430 described above, to generate the raw brace 3D digital model. However, in some non-limiting embodiments of the present technology, the raw brace 3D digital model can be the brace 3D digital model 1102 determined as a result of executing the first step 600 described above.

Thus, in some non-limiting embodiments of the present technology, the processor 550 can be configured to adjust (or otherwise personalize) the raw configuration of the spinal brace 200 determined based on the brace 3D digital model 1102 of the spinal brace 200 to increase the wear comfort and safety thereof during the spine correction treatment. To do so, as will be described in greater detail below, the processor 550 can be configured to apply, to the brace 3D digital model 1102, an optimization algorithm configured to reshape the brace 3D digital model 1102 considering the parameters indicative of the safety and the wear comfort of the spinal brace 200.

To that end, first, according to certain non-limiting embodiments of the present technology, the processor 550 can be configured to define, along a surface of the brace 3D digital model 1102, a plurality of sub-regions. It is not limited how the processor 550 can be configured to define the plurality of sub-regions along the surface of the brace 3D digital model 1102. For example, in some non-limiting embodiments of the present technology, the processor 550 can be configured to determine the plurality of sub-regions as extending along tendons interconnecting muscles of the muscle system (not depicted) of the torso 102 of the subject.

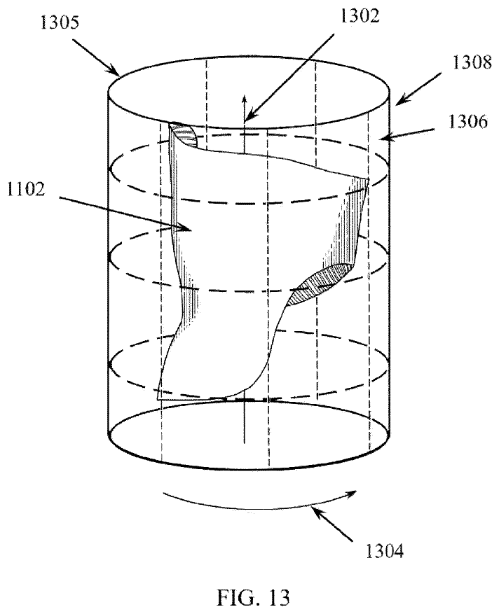

However, in other non-limiting embodiments of the present technology, the processor 550 can be configured to determine the plurality of sub-regions of the brace 3D digital model 1102 differently. With reference to FIG. 13, there is depicted a schematic diagram of a step for sub-dividing, by the processor 550, the surface of the brace 3D digital model 1102 into the plurality of sub-regions, in accordance with certain non-limiting embodiments of the present technology.

More specifically, in accordance with certain non-limiting embodiments of the present technology, the processor 550 can be configured to define, around the brace 3D digital model 1102, a cylindrical coordinate system 1305, such that a longitudinal axis 1302 thereof is aligned with a longitudinal axis (not separately depicted) associated with the brace 3D digital model 1102. For example, the longitudinal axis of the brace 3D digital model 1102 can be determined as coinciding with a longitudinal axis of the subject's body (also known as a “craniocaudal axis”).

Further, the processor 550 can be configured to dissect the brace 3D digital model 1102 along the longitudinal axis 1302 of the cylindrical coordinate system 1305 in a first number of sub-regions 1306 of the plurality of sub-regions. Further, the processor 550 can be configured to dissect the brace 3D digital model 1102 along an azimuth 1304 of the cylindrical coordinate system 1305 in a second number of sub-regions 1308 of the plurality of sub-regions. In some non-limiting embodiments of the present technology, the first number of sub-regions 1306 can be equal to the second number of sub-regions 1308, and can be equal, for example, 6, 7, or 10 sub-regions. However, in other non-limiting embodiments of the present technology, the first number of sub-regions 1306 can be different from the second number of sub-regions 1308.

Also, although, in the embodiments of FIG. 13, each one of the first and second number of sub-regions 1306, 1308 are distributed evenly along the surface of the brace 3D digital model 1102, it may not be the case for each and every non-limiting embodiment of the present technology. In other words, in some regions along the surface of the brace 3D digital model 1102, the processor 550 can be configured to define more sub-regions than in others. For example, such regions of the brace 3D digital model 1102, according to certain non-limiting embodiments of the present technology, may include regions corresponding to a segment of the spine 104 affected by the curvature disorder, such as a thoracic segment of the spine 104 including the misaligned chain 106, as depicted in FIG. 1A.

Also, in additional non-limiting embodiments of the present technology, the processor 550 can be configured to define the plurality of sub-regions within the brace 3D digital model 1102 without determining the cylindrical coordinate system 1305. Instead, in these embodiments, the processor 550 can be configured to remain within a Cartesian coordinate system, such as the reference coordinate system 805 defined by the coronal, sagittal, and transverse planes associated with the subject, as described above with reference to FIG. 8. To that end, the processor 550 can be configured to define the plurality of sub-regions along the axes of the reference coordinate system 805.

The method 1200 hence proceeds to step 1204.

Step 1204: Modulating, by the Processor, an Initial Position of a Given Sub-Region of the Plurality of Sub-Regions to Determine a Surface of the Appliance 3D Digital Model, the Modulating Comprising Executing an Optimization Algorithm; Determining, by the Processor, Based on the Optimized Position for the Given Sub-Region, the Surface of the Appliance 3D Digital Model

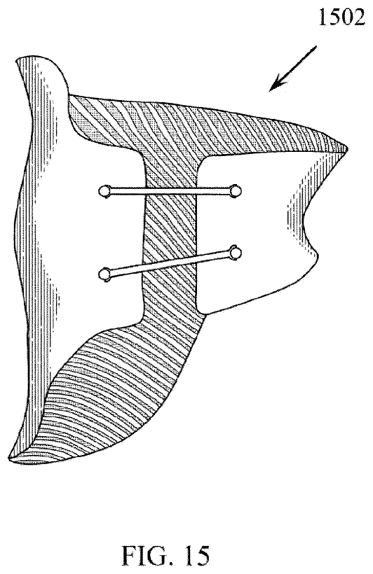

With continued reference to FIG. 13, at step 1204, according to certain non-limiting embodiments of the present technology, the processor 550 can be configured to modulate a position of each one of the plurality of sub-regions of the brace 3D digital model 1102, defined at step 1202, thereby reshaping the surface of the brace 3D digital model 1102 of the spinal brace 200 to generate an optimized 3D digital model thereof (such as an optimized brace 3D digital model 1502 depicted in FIG. 15), as will be described below.

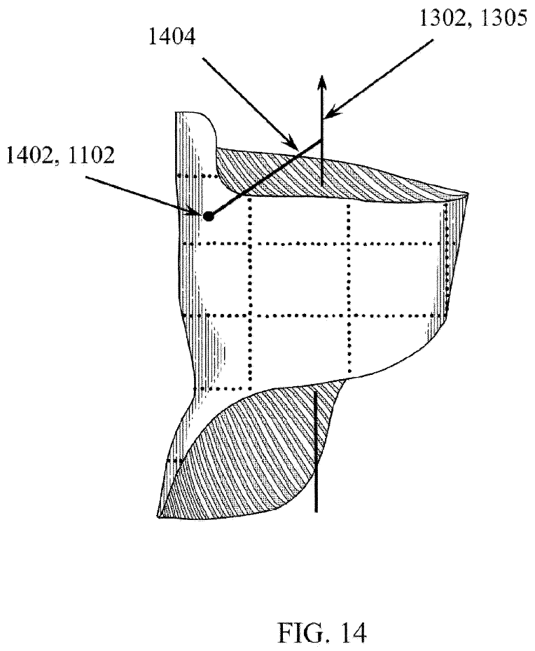

With reference to FIG. 14, there is depicted a schematic diagram explaining how the processor 550 can be configured to modulate a position of a given sub-region 1402 of the plurality of sub-regions of the brace 3D digital model 1102, in accordance with certain non-limiting embodiments of the present technology.

More specifically, according to certain non-limiting embodiments of the present technology, the processor 550 can be configured to modulate an initial value of a respective distance 1404 of the given sub-region 1402 relative to the longitudinal axis 1302 of the cylindrical coordinate system 1305 mentioned above, thereby determining an optimized position for the given sub-region 1402.

In some non-limiting embodiments of the present technology, the processor 550 can be configured to modulate the initial position of the given sub-region 1402 considering an anatomical parameter associated with the torso 102 of the subject when the spinal brace 200 is worn therein. In some non-limiting embodiments of the present technology, the anatomical parameter can comprise an alignment metric for the misaligned chain 106. In these embodiments, the processor 550 can be configured to modulate the initial position of the given sub-region 1402 by optimizing the alignment metric, as will be described below.

In some non-limiting embodiments of the present technology, the anatomical parameter can include a safety parameter. In these embodiments, the processor 550 can be configured to modulate the initial position of the given sub-region 1402 such that the safety parameter is not greater than a safety threshold, as will described further below.

In some non-limiting embodiments of the present technology, while determining the optimized position of the given sub-region 1402 by optimizing the alignment metric, the processor 550 can further be configured to consider the safety parameter such that it is not greater than the safety threshold, as will be described further below.

In some non-limiting embodiments of the present technology, the processor 550 can be configured to determine the initial value of the respective distance 1404 as being a distance value to a predetermined reference vertex (not separately labelled) defining a surface of the given sub-region 1402. Further, the processor 550 can be configured to use a position of the predetermined reference vertex as a proxy for the position of the given sub-region 1402 without considering the surface thereof as a whole, which may help to save computational resources of the processor 550. It is not limited how the processor 550 can be configured to select the predetermined reference vertex along the surface of the given sub-region 1402, and can include, for example, a vertex representative of a center of mass of the given sub-region 1402, a vertex representative of a maximum curvature (either negative or positive) along the surface of the given sub-region 1402, and the like.

Returning to FIGS. 13 and 14, according to certain non-limiting embodiments of the present technology, the alignment metric is indicative of a goal of the spine correction treatment, that is, that the spinal brace 200 manufactured according to the optimized brace 3D digital model 1502 would cause each one of the misaligned chain 106 of vertebrae to move to their respective target positions, associated with their alignment within the spine 104. More specifically, the alignment metric is indicative of a difference between a respective current position of the given vertebra 108 of the misaligned chain 106 and the respective target position 904, corresponding to the normal curvature of the spine 104, as described above. In other words, the alignment metric can be said to be indicative of effectiveness of the spinal brace 200 in causing each one of the misaligned chain 106 to move towards their respective target position.

According to certain non-limiting embodiments of the present technology, to determine the optimized position for the given sub-region 1402, the processor 550 can be configured to iteratively minimize the alignment metric by applying an optimization algorithm. It is not limited how the optimization algorithm can be implemented; and, in specific non-limiting embodiments of the present technology, the optimization algorithm can include a surrogate optimization algorithm. Other non-limiting examples of the optimization can include: (i) Genetic algorithms (GA); (ii) Derivative-free methods for non-linear multivariable function including, for example, a Nelder-Mead simplex optimization algorithm; (iii) Gradient-based non-linear optimization algorithms, including, for example, an interior point optimization algorithm, a trust region optimization algorithm, a sequential quadratic programming (SQP) optimization algorithm, and an active set optimization algorithm; (iv) a Pattern Search optimization algorithm; and (v) a Pattern Swarm optimization algorithm.

According to certain non-limiting embodiments of the present technology, the processor 550 can be configured to determine the respective current position of the given vertebra 108, at a given iteration of the optimization algorithm, by modelling in the torso 3D digital model 702, using principles of the FEA, as described above, a respective in-brace position of the spine 104. The processor 550 can be configured to model the respective in-brace position of the spine 104 by applying, to the torso 3D digital model 702, a respective intermediate configuration of the optimized brace 3D digital model 1502 defined by respective intermediate optimized positions of each one of the plurality of sub-regions, such as the given sub-regions 1402, determined at the given iteration of the optimization algorithm. Further, based on a difference between the respective current position, at the given iteration, and the respective target position of the given vertebra 108, the processor 550 can be configured to determine a respective value of the alignment metric, minimizing which, the processor 550 can further be configured to determine the optimized position for the given sub-region 1402 of the optimized brace 3D digital model 1502.

In some non-limiting embodiments of the present technology, the alignment metric, for the misaligned chain 106, can be determined as an aggregate Root Mean Square Error (RMSE) between respective current positions, at the given iteration of the optimization algorithm, and the respective target positions of each one of the misaligned chain 106. To that end, according to certain non-limiting embodiments of the present technology, the processor 550 can be configured to determine the respective current and target positions by determining respective coordinates values of reference points of each one of the misaligned chain 106 based on the torso 3D digital model 702, described above with reference to FIG. 7, in the reference coordinate system 805. To that end, in these embodiments, the alignment metric can be expressed by a following equation:

$$\sqrt{{\sum }_{i = 1}^{n} [ {( {x}_{2} - {x}_{1} )}_{i}^{2} + {( {y}_{2} - {y}_{1} )}_{i}^{2} + {( {z}_{2} - {z}_{1} )}_{i}^{2} ]} , \qquad ( 3 )$$

where: x₁ and x₂ are X coordinates of the respective current position and the respective target position of the given vertebra 108 of the misaligned chain 106 in the reference coordinate system 805;
   y₁ and y₂ are Y coordinates of the respective current position and the respective target position of the given vertebra 108 of the misaligned chain 106 in the reference coordinate system 805;
   z₁ and z₂ are Z coordinates of the respective current position and the respective target position of the given vertebra 108 of the misaligned chain 106 in the reference coordinate system 805; and
   N is a number of vertebrae in the misaligned chain 106.

However, in other non-limiting embodiments of the present technology, the processor 550 can be configured to determine the alignment metric based on certain anatomical reference values associated with the spine 104, indicative of various curvature disorders of the spine 104. More specifically, in some non-limiting embodiments of the present technology, the alignment metric can be determined as an aggregate difference between certain anatomical reference parameters associated with the spine 104 in states thereof where each one of the plurality of vertebrae is in the respective current position and the respective target position thereof. It is not limited which anatomical reference parameters associated with the spine 104 the processor 550 can be used for defining the alignment metric; however, in some non-limiting embodiments of the present technology, such anatomical reference parameters can include a plurality of anatomical reference angles associated with the spine 104.

With reference to FIGS. 16A and 16B, there are depicted coronal and sagittal views of the spine 104 for determining the plurality of anatomical reference angles associated therewith, in accordance with certain non-limiting embodiments of the present technology.

According to certain non-limiting embodiments of the present technology, some of the plurality of anatomical reference angles associated with the spine 104 can include angles indicative of the scoliosis of the spine 104. For example, a first one of the plurality of anatomical reference angles can include a thoracic Cobb angle 1602. Generally speaking, a Cobb angle is a metric of the curvature disorder of the spine 104 in a given segment thereof, such as one of a cervical segment, a thoracic segment, and a lumbar segment of the spine 104, in one of the anatomical planes associated with the subject. A given Cobb angle, such as the thoracic Cobb angle 1602, can be defined as an angle between lines extending, in the coronal plane associated with the subject, along a superior endplate (not separately labelled) of a superior vertebra (also not separately labelled) and an inferior endplate (not separately labelled) of an inferior vertebra (also not separately labelled), respectively, of the thoracic segment of the spine 104. Thus, using the torso 3D digital model 702, the processor 550 can be configured to determine a value of the thoracic Cobb angle 1602 when each one of the plurality of vertebrae of the spine 104 are in their respective current positions, such as the respective initial position 704 of the given vertebra 108 mentioned above with reference to FIG. 7.

Further, the processor 550 can be configured to cause, in the torso 3D digital model 702, movement of each vertebra of the misaligned chain 106 of vertebrae of the spine 104 from their respective current positions to the respective target positions thereof to determine a value of the thoracic Cobb angle 1602 in the state of the spine 104 where each one of the plurality of vertebrae each vertebra of the misaligned chain 106 of vertebrae are in their respective target positions, associated with the normal curvature of the spine 104 schematically depicted in FIG. 1B.

Further, in some non-limiting embodiments of the present technology, a second one of the plurality of anatomical reference angles can be a lumbar Cobb angle 1604, defined similarly to the thoracic Cobb angle 1602, for the lumbar segment of the spine 104. Thus, similarly, the processor 550 can be configured to determine values of the lumbar Cobb angle 1604 in the states of the spine 104 where each vertebra of the misaligned chain 106 of vertebrae are in their respective current and target positions.

Further, according to some non-limiting embodiments of the present technology, some of the plurality of anatomical reference angles associated with the spine 104 can be indicative of such curvature disorders as a kyphosis and lordosis, for example. To that end, a third and fourth ones of the plurality of anatomical reference angles can be a thoracic kyphosis angle 1608 and a lumbar lordosis angle 1610, whose values can be respectively indicative of the kyphosis and lordosis of the spine 104. As it can be appreciated, the thoracic kyphosis angle 1608 and the lumbar lordosis angle 1610 are defined similarly to the above Cobb angles but in the sagittal view of the spine 104. Thus, the processor 550 can also be configured to determine values of these angles in the states of the spine 104 where each vertebra of the misaligned chain 106 of vertebrae are in their respective current and target positions.

Further, in some non-limiting embodiments of the present technology, a fifth one of the plurality of anatomical reference angles, for the given vertebra 108 of the misaligned chain 106 of vertebrae of the spine 104, can be a respective axial rotation angle (not depicted) thereof in the transverse plane associated with the subject. The processor 550 can be configured to determine values of the respective axial rotation angle associated with the given vertebra 108 in the respective current position, such as the respective initial position 704, and the respective target position thereof. Similarly, the processor 550 can be configured to determine these values for each other one of the misaligned chain 106 of vertebrae.

It should be noted that, in some non-limiting embodiments of the present technology, the processor 550 can be configured to determine values of each one of the plurality of anatomical reference angles in the state of the spine 104 where each one of the misaligned chain 106 of vertebrae is in the respective target position thereof based on reference data associated with other subjects, whose spines correspond to the respective target position of each vertebra of the misaligned chain 106 of vertebrae, such as to the normal curvature of the spine 104.

In other non-limiting embodiments of the present technology, the processor 550 can be configured to determine the above angles analytically. More specifically, the processor 550 can be configured to (i) approximate, such as by using a spline approximation, a curvature of the spine 104, thereby generating a 3D curve; and (ii) determine the above angles based on points of changing curvature of the 3D curve approximating the curvature of the spine 104.

Also, it should be expressly understood that the above plurality of anatomical reference parameters associated with the spine 104 is not exhaustive, and can further include, without limitation, additional anatomical reference parameters in the coronal plane, such as: a Ferguson angle; and a Coronal balance (both angle and distance).

Further, additional anatomical reference parameters in the sagittal plane may include, without limitation: an incidence of a sagittal vertical axis associated with the spine 104; a Pelvic incidence angle; a sacral slope angle; and a pelvic tilt angle.

Further, additional anatomical reference parameters in the traverse plane may include, without limitation: a maximal axial vertebral rotation, determined as across the entire plurality of vertebrae of the spine 104; an average axial vertebral rotation, determined as across the entire plurality of vertebrae of the spine 104; an apical axial rotation of an apical vertebra the misaligned chain 106 of vertebrae; an orientation of plane of maximum curvature of the spine 104; Da Vinci angles;

Other anatomical reference parameters can include, without limitation: a rib hump angle; a Cobb angle in plane of the maximum curvature of the spine 104; a vertebral wedging angle; an apical ratio between left ad right height values the apical vertebra of the spine 104; a Global Alignment and Proportion (GAP) score associated with the spine 104 (in cases where the subject is an adult); a misalignment degree of a sagittal vertical axis (SVA) associated with the spine 104; and a geometric torsion of the spine 104.

According to certain non-limiting embodiments of the present technology, the GAP score can be determined as described in C. Yilgor et al., “GLOBAL ALIGNMENT AND PROPORTION (GAP) SCORE: DEVELOPMENT AND VALIDATION OF A NEW METHOD OF ANALYZING SPINOPELVIC ALIGNMENT TO PREDICT MECHANICAL COMPLICATIONS AFTER ADULT SPINAL DEFORMITY SURGERY,” the content of which is incorporated herein by reference in its entirety.

According to certain non-limiting embodiments of the present technology, after determining the values of each one of the plurality of anatomical reference parameters associated with the spine 104, the processor 550 can further be configured to determine respective difference values between the values of each one of the plurality of anatomical reference parameters in the current (such as the respective in-brace position) and target position of the spine 104, at each iteration of the optimization algorithm. Further, according to certain non-limiting embodiments of the present technology, based on these difference values, the processor 550 can be configured to determine the alignment metric as being an objective function having a following look:

$$OF score = {W}_{in - brace} * [ {W}_{c o r o n a l}^{in - brace} * ( {Δ}_{Cobb M T}^{in - brace} + {Δ}_{Cobb T L L}^{in - brace} ) + {W}_{sagittal}^{in - brace} * ( {Δ}_{TK}^{in - brace} {Δ}_{LL}^{in - brace} ) + {W}_{transverse}^{in - brace} * {Δ}_{Axial rot}^{in - brace} ] , \qquad ( 4 )$$

where:
Δ^{in-brace}_{CobbMT} is a difference between the values of the thoracic Cobb angle 1602 of the spine 104 in states thereof where each one of the misaligned chain 106 of vertebrae is in the respective current position and the respective target position thereof;
Δ^{in-brace}_{CobbTLL} is a difference between the values of the lumbar Cobb angle 1604 of the spine 104 in states thereof where each one of the misaligned chain 106 of vertebrae is in the respective current position and the respective target position thereof;
Δ^{in-brace}_{TK} is a difference between the values of the thoracic kyphosis angle 1606 of the spine 104 in states thereof where each one of the misaligned chain 106 of vertebrae is in the respective current position and the respective target position thereof;
Δ^{in-brace}_{LL} is a difference between the values of the lumbar lordosis angle 1608 of the spine 104 in states thereof where each one of the misaligned chain 106 of vertebrae is in the respective current position and the respective target position thereof;
Δ^{in-brace}_{Axial rot} is an aggregate difference between values of the respective axial rotation angles of each one of the misaligned chain 106 of vertebrae in the respective current and target positions thereof; and
W_{in-brace}, W^{in-brace}_{coronal}, W^{in-brace}_{sagittal}, W^{in-brace}ₜᵣₐₙₛᵥₑᵣₛₑ are predetermined weight coefficients.

According to certain non-limiting embodiments of the present technology, values for the predetermined weight coefficients can be obtained from reference data associated with other subjects having received the spine correction treatment. However, in other non-limiting embodiments of the present technology, the predetermined weight coefficients can be determined for the subject individually, such as by the orthopedic practitioner.

Thus, the processor 550 can be configured to determine the alignment metric associated with the spine 104 based on the plurality of anatomical reference parameters associated therewith, which are indicative of certain curvature disorders of the spine 104. In other words, by optimizing such an alignment metric, the processor 550 can be configured to determine a configuration of the optimized brace 3D digital model 1502 configured to cause each one of the misaligned chain 106 of the plurality of vertebrae of the spine 104 to move to their respective target positions in each anatomical plane associated with the subject, that is, the coronal, sagittal, and transverse planes, allowing for a holistic approach to correcting the curvature of the spine 104.

Thus, with back reference to FIGS. 13, 14, and 15, according to certain non-limiting embodiments of the present technology, having determined the alignment metric as described above, the processor 550 can further be configured to optimize the alignment metric, thereby determining the optimized position for the given sub-region 1402 defining the surface of the optimized brace 3D digital model 1502.

To that end, as mentioned hereinabove, according to certain non-limiting embodiments of the present technology, the processor 550 can be configured to execute the optimization algorithm, which is configured to determine, at each iteration, a respective intermediate position of the given sub-region 1402 defining a respective intermediate configuration of the optimized brace 3D digital model 1502. More specifically, according to these embodiments, the processor 550 executing the optimization algorithm can be configured to execute, at each iteration, the following optimization steps: (i) applying a previous intermediate configuration of the optimized brace 3D digital model 1502, indicative of a then current configuration of the spinal brace 200, from a previous iteration of the optimization algorithm, to the configuration of the torso 3D digital model 702 indicative of the initial configuration of the spine 104 to simulate the application of the then current configuration of the spinal brace 200 to the torso 102; (ii) determining, based on the torso 3D digital model 702, respective stress values imposed onto each vertebra of the misaligned chain 106 by the then current configuration of the spinal brace 200; (iii) determining, based on the respective stress values, the respective current positions of each vertebra of the misaligned chain 106 of vertebrae (that is, the respective in-brace positions thereof); and (iv) based on the respective current positions of the misaligned chain, determining a current value of the alignment metric expressed by any one of Equation (3) or (4). Further, the processor 550 can be configured to minimize, at each iteration of the optimization algorithm, such as by a predetermined step, the current value of the alignment metric, based on which the processor 550 can further be configured to determine the respective intermediate position for the given sub-region 1402 defining the surface of the respective intermediate configuration of the optimized brace 3D digital model 1502.

The processor 550 can be configured to run the optimization algorithm a plurality of iterations, such as hundreds, thousands, or even hundreds of thousands iterations, until a minimum value of the alignment metric is attained, which would correspond to the optimized value of the respective distance 1404 and thus the optimized position of the given sub-region 1402 of the optimized brace 3D digital model 1502.

Also, as it can be appreciated, certain subjects to the spine correction treatment are adolescents, whose skeletal systems are still in a growth phase and thus may change during the spine correction treatment. Thus, in certain non-limiting embodiments of the present technology, it has been appreciated that taking into account a factor of growth of the skeletal system of the subject, when optimizing the raw configuration of the spinal brace 200, would allow applying corrective pressures in desired anatomical locations along the surface of the torso 102. Thus may help further improve the effectiveness of the spine correction treatment and the wear comfort of the spinal brace 200.

Thus, in some non-limiting embodiments of the present technology, the processor 550 can be configured to factor in growth-related changes of the skeletal system of the subject during the spine correction treatment, including at least changes of the spine 104. To do so, according to certain non-limiting embodiments of the present technology, the processor 550 can be configured to (i) obtain the biometrical reference data of the subject, indicative of how structures of the skeletal system thereof, such as the plurality of vertebrae of the spine 104, change in size, such as in height thereof, over a treatment period of the spine correction treatment; (ii) modify, using the obtained reference data, the torso 3D digital model 702, to determine an updated configuration (not depicted) of the spine 104 by a moment of interest during the treatment period; and (iii) determine the optimized position for the given sub-region 1402 based on the torso 3D digital model 702 including an updated configuration of the spine 104.

In some non-limiting embodiments of the present technology, the moment of interest can be determined as being an end of the treatment period of the spine correction treatment, obtained, for example, from the orthopedic practitioner. However, in other non-limiting embodiments of the present technology, where the spine correction treatment includes a plurality of stages, as will be described hereinbelow, the moment of interest can correspond to an end of one of the plurality of stages of spine correction treatment.

As the torso 3D digital model 702 can be a FEM, to determine the updated configuration of the spine 104, at each iteration of optimizing the position of the given sub-region 1402, using the principles of the FEA, the processor 550 can be configured to: (i) determine, based on the torso 3D digital model 702, stress values on epiphyseal growth plates of the given vertebra 108 in a position of the subject unconstrained by the spinal brace 200, considering only the gravity forces acting on the given vertebra 108; (ii) determine, based on the torso 3D digital model 702, stress values on the epiphyseal growth plates of the given vertebra 108 in in the respective in-brace position of the spine 104 by modelling, aside from the gravity forces, forces from the spinal brace 200 that would be manufactured according to the respective intermediate configuration of the optimized brace 3D digital model 1502 at the given iteration; and (iii) apply, based on the stress values, in the torso 3D digital model 702, a thermal expansion of the given vertebral body 108, thereby simulating growth-related changes thereof, such as the changes in height, as mentioned above.

By way of example, to determine the updated configuration of the spine 104, the processor 550 can be configured to simulate the growth-related changes of the given vertebral body 108 in accordance with a following equation:

$$( \begin{matrix}{G}_{L} = {G}_{m} ( 1 + β ( {σ}_{L} - {σ}_{m} ) ) \\ {G}_{R} = {G}_{m} ( 1 + β ( {σ}_{R} - {σ}_{m} )\end{matrix} ) , \qquad ( 5 )$$

where G_{L} is a longitudinal local growth rate applied on a left side of the epiphyseal growth plates of the given vertebra 108;
   G_{R} is a longitudinal local growth rate applied on a right side of the epiphyseal growth plates of the given vertebra 108;
   Gₘ is a baseline vertebral growth rate (which can be, for example, 0.8 mm/year for a thoracic vertebra, and 1.1 mm/year for a lumbar vertebra);
   β is a vertebral bone stress sensitivity factor, can be determined based on reference data associated with subject; for example, β can be determined as being 1.5 MPa⁻¹;
   σ_{L} is a stress applied on the left side of the epiphyseal growth plates;
   σ_{R} is a stress applied on the right side of the epiphyseal growth plates; and
   σₘ is an average stress applied on entire epiphyseal growth plates.

In some non-limiting embodiments of the present technology, the processor 550 can be configured to apply, in the torso 3D digital model 702, the thermal expansion to the given vertebra 108 to simulate the so determined stresses values imposed on the given vertebra in the unconstrained and in the in-brace positions thereof in accordance with Hueter-Volkmann's law describing dependences between the applied stresses to bony structures and growth thereof. In so doing, the determined stress values allow “guiding” the thermal expansion applied to the given vertebra 108 in the torso 3D digital model 702 simulating a more realistic growth thereof under the application of the spinal brace 200.

Further, in these embodiments, to consider the growth-related changes of the skeletal system of the subject, in the alignment metric expressed by Equation (4), the processor 550 can be configured to determine the above difference values of Equation (4) for the states of the spine 104 where each vertebra of the misaligned chain 106 of vertebrae is in a respective future modelled position and the respective target position thereof. According to certain non-limiting embodiments of the present technology, the processor 550 can be configured to determine the respective future modelled position (also referred to herein as “future simulated” position in those embodiments where modelling is performed digitally) as being a position of the given vertebra 108 within the updated configuration of the spine 104 by the moment of interest during the treatment period. In other words, according to certain non-limiting embodiments of the present technology, the respective future modelled position is a position of the given vertebra 108 within the updated configuration of the spine 104 including growth-related changes thereof that have been simulated by the processor 550, in the torso 3D digital model 702 (such as using the principles of the FEA, as described above), given an application of the respective configuration of the spinal brace 200 to the torso 102 of the subject during a treatment period by the moment of interest. In this regard, the respective configuration of the spinal brace 200 is assumed to be produced in accordance with the respective configuration of the appliance 3D digital model 1502 at the given iteration of the optimization algorithm. The respective future modelled position is thus indicative of not only an intermediate position of the given vertebra 108 by the moment of interest, but also changed dimensions thereof by the moment of interest during the spine correction treatment.

Thus, the objective function of Equation (4) including the growth-related changes of the skeletal system factored therein can have a following look:

$$OF score = {W}_{in - brace} * [ {W}_{coronal}^{in - brace} * ( {Δ}_{Cobb M T}^{in - brace} + {Δ}_{Cobb T L L}^{in - brace} ) + {W}_{sagittal}^{in - brace} * ( {Δ}_{TK}^{in - brace} + {Δ}_{LL}^{in - brace} ) + {W}_{transverse}^{in - brace} * {Δ}_{Axial rot}^{in - brace} ] + W * [ {W}_{coronal}^{growth} * ( {Δ}_{Cobb M T}^{growth} + {Δ}_{Cobb T L L}^{growth} ) + {W}_{sagittal}^{growth} * ( {Δ}_{TK}^{growth} + {Δ}_{LL}^{growth} ) + {W}_{transverse}^{growth} * {Δ}_{Axial rot}^{growth} ] , \qquad ( 6 )$$

where:
Δ^{growth}_{CobbMT} is a difference between the values of the thoracic Cobb angle 1602 of the spine 104 in states thereof where each one of the misaligned chain 106 of vertebrae is in the respective future modelled position and the respective target position thereof;
Δ^{growth}_{CobbTLL} is a difference between the values of the lumbar Cobb angle 1604 of the spine 104 in states thereof where each one of the misaligned chain 106 of vertebrae is in the respective future modelled position and the respective target position thereof;
Δ^{growth}_{TK} is a difference between the values of the thoracic kyphosis angle 1606 of the spine 104 in states thereof where each one of the misaligned chain 106 of vertebrae is in the respective future modelled position and the respective target position thereof;
Δ^{growth}_{LL} is a difference between the values of the lumbar lordosis angle 1608 of the spine 104 in states thereof where each one of the misaligned chain 106 of vertebrae is in the respective future modelled position and the respective target position thereof;
Δ^{growth}_{Axial rot} is an aggregate difference between values of the respective axial rotation angles of each one of the misaligned chain 106 of vertebrae in the respective future modelled position and the respective target position thereof; and
W_{growth}, W^{growth}_{coronal}, W^{growth}_{sagittal}, W^{growth}ₜᵣₐₙₛᵥₑᵣₛₑ are predetermined weight coefficients.

Similar to Equation (4), values for other predetermined weight coefficients used in Equation (6) can be obtained from the reference data associated with other subjects having received the spine correction treatment or from the orthopedic practitioner. Needles to mention, the processor 550 can be configured to optimize the alignment metric according to Equation (6) in a similar manner described above with respect to the alignment metric according to Equations (3) and (4) to determine the optimized position for the given sub-region 1402 defining the surface of the optimized brace 3D digital model 1502 considering the growth-related changes of the subject.

In yet other non-limiting embodiments of the present technology, the alignment metric can be expressed by the objective function having a following configuration:

$$OF score = {W}_{in - brace} * ( {W}_{corMT} \vert \frac{Cobb {MT}_{in - brace}}{Cobb {MT}_{INI}} \vert + {W}_{corTLL} \vert \frac{Cobb {TLL}_{in - brace}}{Cobb {TLL}_{INI}} \vert + {W}_{sagTK} \vert \frac{\vert {TK}_{in - brace} \vert - \vert {TK}_{N} \vert}{\vert {TK}_{INI} \vert - \vert {TK}_{N} \vert} \vert + {W}_{sagLL} \vert \frac{\vert {LL}_{in - brace} \vert - \vert {LL}_{N} \vert}{\vert {LL}_{INI} \vert - \vert {LL}_{N} \vert} \vert + {W}_{transverse MT} \vert \frac{AVR {MT}_{in - brace}}{AVR {MT}_{INI}} \vert + {W}_{transverse TLL} \vert \frac{AVR {TLL}_{in - brace}}{AVR {TLL}_{INI}} \vert ) + {W}_{growth} * ( {W}_{corMT} \vert \frac{Cobb {MT}_{growth}}{{CobbMT}_{INI}} \vert + {W}_{corTLL} \vert \frac{CobbTL {L}_{growth}}{CobbTL {L}_{INI}} \vert + {W}_{sagTK} \vert \frac{\vert {TK}_{growth} \vert - \vert {TK}_{N} \vert}{\vert {TK}_{INI} \vert - \vert {TK}_{N} \vert} \vert + {W}_{sagLL} \vert \frac{\vert {LL}_{growth} \vert - \vert {LL}_{N} \vert}{\vert {LL}_{INI} \vert - \vert {LL}_{N} \vert} \vert + {W}_{transverse MT} \vert \frac{AVR {MT}_{growth}}{AVR {MT}_{INI}} \vert + {W}_{transverse TLL} \vert \frac{AVR {TLL}_{growth}}{AVR {TLL}_{INI}} \vert ) , \qquad ( 7 )$$

where Cobb MT_{in-brace} is a value of the thoracic Cobb angle 1602 of the spine 104 in the state thereof where each one of the misaligned chain 106 of vertebrae is in the respective current position;
Cobb MT_{INI} is a value of the thoracic Cobb angle 1602 of the spine 104 in the state thereof where each one of the misaligned chain 106 of vertebrae is in the respective initial position;
Cobb TLL_{in-brace} is a value of the lumbar Cobb angle 1604 of the spine 104 in the state thereof where each one of the misaligned chain 106 of vertebrae is in the respective current position;
Cobb TLL_{INI} is a value of the lumbar Cobb angle 1604 of the spine 104 in the state thereof where each one of the misaligned chain 106 of vertebrae is in the respective initial position;
TK_{in-brace} is a value of the thoracic kyphosis angle 1606 of the spine 104 in the state thereof where each one of the misaligned chain 106 of vertebrae is in the respective current position;
TK_{INI} is a value of the thoracic kyphosis angle 1606 of the spine 104 in the state thereof where each one of the misaligned chain 106 of vertebrae is in the respective initial position;
TK_{N} is a value penalizing TK_{in-brace} if it is outside the predetermined thoracic kyphosis angle normal range;
LL_{in-brace} is a value of the lumbar lordosis angle 1608 of the spine 104 in the state thereof where each one of the misaligned chain 106 of vertebrae is in the respective current position;
LL_{INI} is a value of the lumbar lordosis angle 1608 of the spine 104 in the state thereof where each one of the misaligned chain 106 of vertebrae is in the respective initial position;
LL_{N} is a value penalizing LL_{in-brace} if it is outside the predetermined lumbar lordosis angle normal range;
AVR MT_{in-brace} is a value of an axial rotation angle of an apical vertebra of the main thoracic segment of the spine 104 in the state thereof where each one of the misaligned chain 106 of vertebrae is in the respective current position;
AVR MT_{INI} is a value of the axial rotation angle of the apical vertebra of the main thoracic segment of the spine 104 in the state thereof where each one of the misaligned chain 106 of vertebrae is in the respective initial position;
AVR TLL_{in-brace} is a value of an axial rotation angle of an apical vertebra of a thoracolumbar segment of the spine 104 in the state thereof where each one of the misaligned chain 106 of vertebrae is in the respective current position;
AVR TLL_{INI} is a value of the axial rotation angle of the apical vertebra of the thoracolumbar segment of the spine 104 in the state thereof where each one of the misaligned chain 106 of vertebrae is in the respective initial position;
Cobb MT_{growth} is a value of the thoracic Cobb angle 1602 of the spine 104 in the state thereof where each one of the misaligned chain 106 of vertebrae is in the respective future modelled position;
Cobb TLL_{growth} is a value of the lumbar Cobb angle 1604 of the spine 104 in the state thereof where each one of the misaligned chain 106 of vertebrae is in the respective future modelled position;
TK_{growth} is a value of the thoracic kyphosis angle 1606 of the spine 104 in the state thereof where each one of the misaligned chain 106 of vertebrae is in the respective future modelled position;
LL_{growth} is a value of the lumbar lordosis angle 1608 of the spine 104 in the state thereof where each one of the misaligned chain 106 of vertebrae is in the respective future modelled position;
AVR MT_{growth} is a value of the axial rotation angle of the apical vertebra of the main thoracic segment of the spine 104 in the state thereof where each one of the misaligned chain 106 of vertebrae is in the respective future modelled position;
AVR TLL_{growth} is a value of the axial rotation angle of the apical vertebra of the thoracolumbar segment of the spine 104 in the state thereof where each one of the misaligned chain 106 of vertebrae is in the respective future modelled position; and
W_{corMT}, W_{corTLL}, W_{sagTK}, W_{sagLL}, W_{transverse MT}, W_{transverse TLL}, are predetermined weight coefficients.

In some non-limiting embodiments of the present technology, TK_{N} can be equal: (i) TK_{in-brace} if TK_{in-brace} falls within the predetermined thoracic kyphosis angle normal range; (ii) 20 degrees if TK_{in-brace} <20 degrees; and (iii) 40 degrees if TK_{in-brace} >40 degrees. Thus, if TK_{in-brace} falls within the predetermined thoracic kyphosis angle normal range, which corresponds to the normal curvature of the spine 104, a sagittal term of Equation (7) following the predetermined weight value W_{sagTK} will be reduced to 0 (zero). For example, the predetermined thoracic kyphosis angle normal range for the thoracic kyphosis angle 1606 is from 20 to 40 degrees.

Further, in some non-limiting embodiments of the present technology, e, LL_{N} can be equal: (i) LL_{in-brace} if LL_{in-brace} falls within the predetermined lumbar lordosis angle normal range; (ii) 30 degrees if LL_{in-brace}<30 degrees; and (iii) 60 degrees if LL_{in-brace}>60 degrees. Thus, if LL_{in-brace} falls within this the predetermined lumbar lordosis angle normal range, which corresponds to the normal curvature of the spine 104, a sagittal term of Equation (7) following the predetermined weight value W_{sagLL} will be reduced to 0 (zero). For example, the predetermined lumbar lordosis angle normal range for the lumbar lordosis angle 1608 can be from 30 to 60 degrees. More details on how the values of the thoracic kyphosis angle 1606 and the lumbar lordosis angle 1608 corresponding to the normal ranges thereof can be determined can be found, for example, in “RESTORATION OF THORACIC KYPHOSIS AFTER OPERATIVE TREATMENT OF ADOLESCENT IDIOPATHIC SCOLIOSIS: A MULTICENTER COMPARISON OF THREE SURGICAL APPROACHES” by D. J. Sucato et al., content of which is incorporated herein by reference in its entirety.

As mentioned above, the predetermined weight coefficients can be obtained from the reference data associated with other subjects having received the spine correction treatment or from the orthopedic practitioner. However, by way of example only, and in now way as a limitation, W^{in-brace} can be 5; W^{growth} can be 10; W_{corMT} can be equal to W_{corTLL}, which can have a value of 2; W_{sagTK} can be equal to W_{sagLL}, which can have a value of 1; and W transverse MT can be equal to W_{transverse TLL}, which can have a value of 1.

Further, it should be expressly understood that the configurations of the objective function expressed by Equations (4), (6), and (7) are given as non-limiting examples only. In other non-limiting embodiments of the present technology, certain anatomical reference parameters used for constructing the objective function expressed by the above Equations can be replaced by other parameters representative of the misalignment of the spine 104 in a respective one of the coronal, sagittal, and traverse planes associated with the subject. Also, in some non-limiting embodiments of the present technology, at least one term of any one of Equations (4), (6), and (7) can be omitted.

For example, instead of the values associated with any one of the thoracic Cobb angle 1602 and the lumbar Cobb angle 1604, a respective value of the Fergusson angle can be used. In another example, instead of the values of the axial rotation of the apical vertebra either of the main thoracic or thoracolumbar sections of the spine, other anatomical reference parameters indicative of the misalignment of the spine 104 in the transverse plane can be used, such as one of: the maximal axial vertebral rotation; and the average axial vertebral rotation, as an example.

In yet other example, instead of the values of the thoracic kyphosis angle 1606 and the lumbar lordosis angle 1608 associated with the spine 104, in the above Equations, values of the GAP score (in cases where the subject to the spine correction treatment is an adult) or values of the misalignment degree associated with the spine 104 can be used.

In additional non-limiting embodiments of the present technology, the processor 550 can further be configured to factor in a compliance rate of the subject to wearing the spinal brace 200 in the course of the spine correction treatment. More specifically, the processor 550 can be configured to simulate, in the torso 3D digital model 702, the growth-related changes of the spine 104 taking into account how long the subject will be wearing the spinal brace 200 during each 24 hours. For example, in some embodiment of the present technology, the compliance coefficient can be predetermined as being 0.63. However, other values the compliance coefficient can take include 0.5, 0.75, 0.9, or even 1.0. Thus, to factor the compliance coefficient in the simulation of the growth-related changes of the given vertebra 108, the processor 550 can be configured to proportionally decrease the stress values imposed on the given vertebra 108 in the in-brace position, thereby correcting an amount of the thermal expansion applied thereto to simulate the growth-related changes of the given vertebra 108. This may provide for even more accurate determining of the updated configuration of the spine 104.

More details on simulating the growth-related changes of the given vertebra 108 can be found, for example, in an article entitled “Surgical Planning and Follow-up of Anterior Vertebral Body Growth Modulation in Pediatric Idiopathic Scoliosis Using a Patient-Specific Finite Element Model Integrating Growth Modulation”, authored by Cobetto et al., and published by Spine Deformity Journal on Nov. 10, 2017, the content of which is incorporated herein by reference in its entirety.

Further, in other non-limiting embodiments of the present technology, instead of considering the alignment metric as described above, the processor 550 can be configured to modulate the initial position of the given sub-region 1402 considering the safety of the spine correction treatment. More specifically, in these embodiments, using the optimization algorithm described above, the processor 550 can be configured to iteratively optimize the initial value of the respective distance 1404 of the given sub-region 1402 such that the safety parameter does not exceed a safety threshold.

According to certain non-limiting embodiments of the present technology, the safety parameter can include, without limitation, at least one of: a contact skin pressure on the torso 102 of the subject; a distance between the spinal brace 200 and an outer surface of the torso 102 of the subject when the spinal brace 200 is applied thereto; a minimum clearance distance between the spinal brace 200 and the pelvis of the subject when the spinal brace 200 is applied to the torso 102 of the subject; a minimum clearance distance between the spinal brace 200 and breasts of the subject when the spinal brace 200 is applied to the torso 102 of the subject; smoothness parameter of a surface of the spinal brace 200; a maximum stress and/or strain inside the spinal brace 200; a maximum stress and/or strain inside the torso (soft tissues and/or skeleton, including the spine 104 of the subject).

As it can be appreciated, a given value of the safety threshold depends on a respective configuration of the safety parameter, and can include a vector of respective predetermined threshold values for each one of the above characteristics of the safety parameter.

It should be noted that, in some non-limiting embodiments of the present technology, the respective predetermined threshold values for the safety parameter can be obtained from reference data associated with other subjects that have received the spine correction treatment. However, in other non-limiting embodiments of the present technology, the respective predetermined threshold value can be determined considering anatomical specifics of the subject, such as an age, a gender, a weight, a height, a body mass index (BMI), and the like, and/or medical history of the subject, such as deformed or tilted pelvis, history of spine injuries, or current state of the inner organs, and the like. Also, in some non-limiting embodiments of the present technology, the respective predetermined threshold value can be determined based on subject's individual sensations from wearing the spinal brace 200, such as rubbing or excessive compression along at least one of the plurality of sub-regions, and the like.

Further, according to yet other non-limiting embodiments of the present technology, the processor 550 can be configured to consider the effectiveness of the spine correction treatment, represented by the alignment metric, and the safety of the spine correction treatment, represented by the safety parameter simultaneously. More specifically, in these embodiments, while optimizing the alignment metric described above, the processor 550 can be configured to consider the safety parameter as a safety constraint to minimizing the alignment metric. In other words, by considering the safety parameter while optimizing the alignment metric, the processor 550 can be configured to determine whether a given configuration of the optimized brace 3D digital model 1502 enables to manufacture the respective configuration of the spinal brace 200 that would be safe to the subject, that is, would not cause damage to subject's health and/or wear discomfort, such as pain or excess pressure, from applying the spinal brace 200 during the spine correction treatment.

In other words, the processor 550 can be configured to optimize the alignment metric such that the safety parameter is no greater than a safety threshold, which is indicative of the potential damage to the subject's health and/or wear discomfort.

Thus, in these embodiments, while modulating the initial value of the respective distance 1404 of the given sub-region 1402, as described above, to minimize the alignment metric, the processor 550 can be configured to monitor, using the torso 3D digital model 702, at each iteration of the optimization algorithm, the safety parameter such that it does not exceed the safety threshold. More specifically, in these embodiments, at each iteration, the processor 550 can be configured to determine if the safety parameter is exceeded; and if not, the processor 550 can be configured to proceed to a next iteration of the optimization algorithm to further minimize the alignment metric.

Further, in some non-limiting embodiments of the present technology, the optimized brace 3D digital model 1502 can have a single configuration for manufacturing a single respective configuration of the spinal brace 200 to be applied to the torso 102 of the subject during the entire duration of the spine correction treatment. However, given a current value of the safety threshold value predetermined for the safety parameter, the configuration of the spinal brace 200 manufactured according to the optimized brace 3D digital model 1502 may be incapable of causing each one of the misaligned chain 106 of vertebrae to move to the respective target position in a single iteration. In other words, the safety constrains imposed onto the configuration of the spinal brace 200 may affect its effectiveness. In another example, the optimization algorithm configured to minimize the alignment metric, thereby determining the respective optimized position of each one of the plurality of sub-regions defining the surface of the optimized brace 3D digital model 1502 does not converge to the minimum value of the alignment metric given the current value of the safety threshold value.

To that end, as mentioned hereinabove, according to certain non-limiting embodiments of the present technology, the spine correction treatment can include the plurality of stages dividing the treatment period into a plurality of treatment intervals. A given stage of the plurality of stages can include applying, for a respective treatment interval, a respective configuration of the spinal brace 200 causing at least some of the misaligned chain 106 of vertebrae to move to their respective intermediate target position, such that, at an end of a last one of the plurality of stages, each vertebra of the misaligned chain 106 attains their respective target positions.

Accordingly, to determine the respective configuration of the spinal brace 200, the processor 550 can be configured to determine the respective configuration of the optimized brace 3D digital model 1502 applying one of the approaches described above, based on the respective intermediate target position associated with given stage of the spine correction treatment.

It should be noted that, in some non-limiting embodiments of the present technology, the spine correction treatment can include causing each vertebra of the misaligned chain 106 to move to their respective intermediate target position during each one of the plurality of stages.

However, in other non-limiting embodiments of the present technology, different number of vertebrae of the misaligned chain 106 can be planned to be moved during each one of the plurality of stages of the spine correction treatment.

Further, in some non-limiting embodiments of the present technology, each one of the plurality of treatment intervals associated with the respective stage of the spine correction treatment can have an equal duration. However, in other non-limiting embodiments of the present technology, at least some of the plurality of treatment intervals making up the treatment period can be of different durations.

The method 1200 hence advances to step 1206.

### Step 1206: Storing, by the Processor, in a Memory of the Computer System, Data Indicative of the Appliance 3D Digital Model

At step 1206, according to certain non-limiting embodiments of the present technology, the processor 550 can be configured to store the optimized brace 3D digital model 1502 representing the optimized configuration of the spinal brace 200 in the internal memory of the computing environment 540. As it can be appreciated, the processor 550 can be configured to execute this step similarly to step 612 of the method 600.

Also, as further described above with respect to step 612 of the method 600, the processor 550 can be configured to cause display of the optimized brace 3D digital model 1502, for example, in the screen 422, for presentation thereof to the orthotic practitioner or the subject.

Additionally or alternatively, at step 1206, according to certain non-limiting embodiments of the present technology, as described further above with respect to step 612 of the method 600, the processor 550 can be configured to cause the forming subsystem 440 to manufacture the optimized configuration of the spinal brace 200 according to the optimized brace 3D digital model 1502.

The method 1200 hence terminates.

Thus, certain non-limiting embodiments of the method 1200 allow determining an optimized 3D digital model of the spinal brace 200 allowing not only implementing the goals of the spine correction treatment causing the misaligned vertebra to move towards their target positions within the spine 104, but also taking into account factors of the safety, wear comfort, and growth of the skeletal system of the subject during the implementation of the spine correction treatment, which can provide for the improved efficacy thereof.

In some non-limiting embodiments of the present technology, the processor 550 can be configured to determine the optimized configuration of the spinal brace 200 by sequentially executing the method 600 and the method 1200.

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A computer-implementable method of generating a model of a spine correction appliance to be worn around a torso of a subject for correcting a misalignment in a chain of adjacent vertebrae of a spine of the subject from an initial position to a target position, the method comprising:
obtaining a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin surface topography of the torso;
obtaining a raw appliance 3D digital model of the spine correction appliance,
the raw appliance 3D digital model having been determined such that the spine correction appliance, manufactured according thereto and worn by the subject, causes at least one of the chain of adjacent vertebrae to displace from the respective initial position towards the respective target position thereof;
the raw appliance 3D digital model being sub-divided into a plurality of sub-regions;
modulating an initial position of a given sub-region of the plurality of sub-regions to determine a surface of the appliance 3D digital model, the modulating comprising executing an optimization algorithm, the executing comprising:
iteratively optimizing the initial position of the given sub-region considering an anatomical parameter associated with the torso of the subject when wearing the spine correction appliance, thereby determining, based on the torso 3D digital model, an optimized position for the given sub-region of the raw appliance 3D digital model,
determining, based on the optimized position for the given sub-region, the surface of the appliance 3D digital model; and
storing data indicative of the appliance 3D digital model.

2. The method of claim 1, further comprising determining the raw appliance 3D digital model, the determining comprising:
determining, in the torso 3D digital model, for the given vertebra, a transposed position thereof,
the determining comprises an at least partial mirroring, in the torso 3D digital model, the respective initial position of the given vertebra relative to an anatomical sagittal plane associated with the subject; and
determining, based on the transposed position of the given vertebra, within the torso 3D digital model, a modulated surface topography of the torso corresponding to the transposed position of the given vertebra, the modulated skin surface topography defining an inner surface of the spine correction appliance; and
based on the modulated surface topography of the torso, determining the raw appliance 3D digital model of the spine correction appliance to be applied to the torso of the subject to cause the given vertebra to displace from the initial position towards the transposed position.

3. The method of claim 1, wherein:
the raw appliance 3D digital model is associated with a predetermined longitudinal axis; and
the modulating the initial position of the given sub-region comprises modulating a respective distance of the given sub-region relative to a longitudinal axis of a cylindrical coordinate system defined around the raw appliance 3D digital model such that the longitudinal axis of the cylindrical coordinate system is aligned with the predetermined longitudinal axis of the raw appliance 3D digital model.

4. (canceled)

5. The method of claim 4, further comprising sub-dividing the raw appliance 3D digital model in the plurality of sub-regions, and wherein the sub-dividing the raw appliance 3D digital model comprises:
dissecting the surface of the raw appliance 3D digital model along an azimuth of the cylindrical coordinate system into a first number of sub-regions; and
dissecting the surface of the raw appliance 3D digital model along the longitudinal axis of the cylindrical coordinate system into a second number of sub-regions.

6. The method of claim 1, wherein the torso 3D digital model is a finite element model-(FEM).

7. The method of claim 1, wherein:
the anatomical parameter comprises an alignment metric that is indicative of a respective difference value between (i) a respective current position, at a given iteration of the optimization algorithm, and (ii) the respective target position of a given one of the chain of adjacent vertebrae of the spine; and
the iteratively optimizing comprises minimizing the alignment metric.

8. The method of claim 7, further comprising:
acquiring a treatment period for correcting the misalignment of the chain of adjacent vertebrae;
determining, based on the torso 3D digital model, an updated configuration of the spine at an end of the treatment period, the updated configuration being indicative of how the given vertebral body will change in height over the treatment period;
and wherein the executing the optimization algorithm comprises:
iteratively optimizing the initial position of the given sub-region by minimizing the alignment metric, thereby determining, based on the torso 3D digital model including the updated configuration of the spine, the optimized position for the given sub-region of the raw appliance 3D digital model.

9. The method of claim 7, wherein the alignment metric comprises one or more anatomical parameters which are representative of an alignment of the spine.

10. The method of claim 1, wherein the minimizing the alignment metric is executed such that a safety parameter is not greater than a safety threshold, and wherein the safety parameter is one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance.

11. The method of claim 1, wherein:
the anatomical parameter comprises a safety parameter,
the safety parameter comprising one or more of: a contact skin pressure on the torso of the subject; a distance between the spine correction appliance and an outer surface of the torso of the subject when the spine correction appliance is applied thereto; a clearance distance between the spine correction appliance and a pelvis of the subject when the spine correction appliance is applied to the torso of the subject; a clearance distance between the spine correction appliance and the breasts of the subject when the spine correction appliance is applied to the torso of the subject; smoothness parameter of the surface of the appliance 3D digital model; a stress and/or strain applied to the spine of the subject by the spine correction appliance; and
the iteratively optimizing is executed such that the safety parameter is not greater than a safety threshold.

12. The method of claim 1, wherein the spine correction treatment comprises a plurality of stages to be implemented over the treatment period, each stage having a respective treatment interval,
a given stage of the plurality of stages comprising applying, for the respective treatment interval, a respective configuration of the spine correction appliance to the torso of the subject causing at least one of the chain of adjacent vertebrae to move towards the respective target position.

13. The method of claim 1, wherein the respective target position of the given vertebra is a position thereof within the spine having a normal curvature.

14. The method of claim 1, further comprising causing display of the appliance 3D digital model.

15. The method of claim 1, further comprising causing the manufacturing the spine correction appliance according to the appliance 3D digital model.

16. The method of claim 15, wherein the manufacturing comprises 3D printing the spine correction appliance.

17. A system for generating a model of a spine correction appliance to be worn around a torso of a subject for correcting a misalignment in a chain of adjacent vertebrae of a spine of the subject from an initial position to a target position, the system including at least one processor and at least one non-transitory computer-readable memory storing instruction, which, when executed by the at least one processor cause the system to:
obtain a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current skin surface topography of the torso;
obtain a raw appliance 3D digital model of the spine correction appliance, the raw appliance 3D digital model,
the raw appliance 3D digital model having been determined such that the spine correction appliance, manufactured according thereto and worn by the subject, causes at least one of the chain of adjacent vertebrae to displace from the respective initial position the position towards the respective target position thereof;
the raw appliance 3D digital model being sub-divided into a plurality of sub-regions;
modulate an initial position of a given sub-region of the plurality of sub-regions to determine a surface of the appliance 3D digital model, by executing an optimization algorithm, the executing comprising:
iteratively optimizing the initial position of the given sub-region considering an anatomical parameter associated with the torso of the subject when wearing the spine correction appliance, thereby determining, based on the torso 3D digital model, an optimized position for the given sub-region of the raw appliance 3D digital model,
determine, based on the optimized position for the given sub-region, the surface of the appliance 3D digital model; and
store, in the non-transitory computer-readable memory, data indicative of the appliance 3D digital model.

18. The system of claim 17, wherein the at least one processor further causes the system to determine the raw appliance 3D digital model, by:
determining in the torso 3D digital model, for the given vertebra, a transposed position thereof,
the determining comprises an at least partial mirroring, in the torso 3D digital model, the respective initial position of the given vertebra relative to an anatomical sagittal plane associated with the subject; and
determining, based on the transposed position of the given vertebra, within the torso 3D digital model, a modulated surface topography of the torso corresponding to the transposed position of the given vertebra, the modulated surface topography defining an inner surface of the spine correction appliance; and
based on the modulated surface topography of the torso, determining the raw appliance 3D digital model of the spine correction appliance to be applied to the torso of the subject to cause the given vertebra to displace from the initial position towards the transposed position.

19. (canceled)

20. The system of claim 17, wherein the at least one processor further causes the system to sub-divide the raw appliance 3D digital model in the plurality of sub-regions, wherein to sub-divide the raw appliance 3D digital model, the at least one processor causes the system to:
dissect the surface of the raw appliance 3D digital model along an azimuth of the cylindrical coordinate system into a first number of sub-regions; and
dissect the surface of the raw appliance 3D digital model along the longitudinal axis of the cylindrical coordinate system into a second number of sub-regions.

21-25. (canceled)

26. The method of claim 1, further comprising determining the raw appliance 3D digital model, the determining comprising:
determining, in the torso 3D digital model, for the given vertebra, a transposed position thereof,
wherein the determining the transposed position is based on determining if the given vertebra in the torso 3D digital model is misaligned from the respective target position;
determining, based on the transposed position of the given vertebra, within the torso 3D digital model, a modulated surface topography of the torso corresponding to the transposed position of the given vertebra, the modulated surface topography defining an inner surface of the spine correction appliance; and
based on the modulated surface topography of the torso, determining the raw appliance 3D digital model of the spine correction appliance to be applied to the torso of the subject to cause the given vertebra to displace from the initial position towards the transposed position.

27. A computer-implementable method of generating a model of a spine correction appliance to be worn around a torso of a subject for correcting a misalignment in a chain of adjacent vertebrae of a spine of the subject from an initial position to a target position, the method comprising:
obtaining a torso 3D digital model of a torso of the subject, the torso 3D digital model being representative of (i) the spine of the subject including a plurality of vertebrae, a given vertebra of the plurality of vertebrae being associated with a respective initial position and a respective target position; and (ii) a current surface topography of the torso;
obtaining a raw appliance 3D digital model of the spine correction appliance,
the raw appliance 3D digital model having been determined based on an at least partial mirroring in the torso 3D digital model of the respective initial position of the given vertebrae relative to an anatomical plane, or based on determining if the given vertebra in the torso 3D digital model is misaligned from the respective target position, such that the spine correction appliance, manufactured according thereto and worn by the subject, causes at least one of the chain of adjacent vertebrae to displace from the respective initial position towards the respective target position thereof;
modulating an initial position of the raw appliance 3D digital model, the modulating comprising executing an optimization algorithm, the executing comprising:
iteratively optimizing the initial position of the raw appliance 3D digital model considering an anatomical parameter associated with the torso of the subject when wearing the spine correction appliance, thereby determining, based on the torso 3D digital model, an optimized position for the raw appliance 3D digital model,
determining, based on the optimized position, the surface of the appliance 3D digital model; and
causing the manufacturing the spine correction appliance according to the determined surface of the appliance 3D digital model.
